(19) United States
(12) Patent Application Publication
(10) Pub. No.: US 2025/0369046 A1
(43) Pub. Date: Dec. 4, 2025

(54) APTAMER DETECTION TECHNIQUES

(71) Applicant: Illumina, Inc., San Diebo, CA (US)

(72) Inventors: Bryan Anstead, San Diego, CA (US); Carlo Randise-Hinchliff, San Diego, CA (US); Kuanlin Wu, San Diego, CA (US); Anthony Chau, San Diego, CA (US); Steven Norberg, San Diego, CA (US); Nithya Subramanian, Cambridge (GB); Andrew Slatter, Cambridge (GB); Yang Cao, Cambridge (GB); Stephane Emond, Cambridge (GB); Miguel Angel Aleman Garcia, Cambridge (GB); Brian Tenner, San Diego, CA (US); Anastasia Orme, Cambridge (GB)

(21) Appl. No.: 18/876,448

(22) PCT Filed: Mar. 8, 2024
(86) PCT No.: PCT/US2024/019080

§ 371 Date: Dec. 18, 2024

Related U.S. Application Data
(60) Provisional application No. 63/489,609, filed on Mar. 10, 2023.
(60) Provisional application No. 63/517,053, filed on Aug. 1, 2023.
(60) Provisional application No. 63/580,631, filed on Sep. 5, 2023.

(51) Int. Cl. C12Q 1/6874; C12N 15/10; C12Q 1/6855; C12Q 1/6876

(52) U.S. Cl. CPC C12Q 1/6874; C12N 15/1065; C12Q 1/6855; C12Q 1/6876

(57) ABSTRACT

Aptamer detection techniques are described that may include aptamer modification to facilitate incorporation of adapter sequences. In an embodiment, a 3′ end of an aptamer may be modified by deprotection and subsequent ligation to the deprotected 3′ end or extension of the deprotected 3′ end. The modifications at the 3′ end of the adaptor may include adaptor sequences used for library preparation of a sequencing library.

### REFERENCE TO ELECTRONIC SEQUENCE LISTING

The application contains a Sequence Listing which has been submitted electronically in .XML format and is hereby incorporated by reference in its entirety. Said .XML copy, created on Mar. 8, 2024, is named “ilum0133PCT.xml” and is 30,081 bytes in size. The sequence listing contained in this .XML file is part of the specification and is hereby incorporated by reference herein in its entirety.

### BACKGROUND

The disclosed technology relates generally to aptamer detection and/or identification techniques that may be used in conjunction with an aptamer-based assay. In particular, the technology disclosed relates to direct or indirect aptamer detection in conjunction with an aptamer-based assay.

The subject matter discussed in this section should not be assumed to be prior art merely as a result of its mention in this section. Similarly, a problem mentioned in this section or associated with the subject matter provided as background should not be assumed to have been previously recognized in the prior art. The subject matter in this section merely represents different approaches, which in and of themselves can also correspond to implementations of the claimed technology.

Protein expression patterns help define a cell's identity and state. RNA transcripts are often used as a surrogate for protein expression, but the relationship between abundance of proteins and mRNA is not one-to-one. There are differences caused by regulation of posttranscriptional, translational and protein degradation. Therefore, direct nucleic acid sequencing of RNA transcripts may not provide an accurate estimation of protein expression.

Aptamers are nucleic acids that bind to molecular targets, such as proteins, with high affinity and specificity. Advancements in aptamer selection and design include Systematic Evolution of Ligands by EXponential enrichment (SELEX). In SELEX, high affinity nucleic acids for different analytes of interest can be isolated from a combinatorial library, permitting high throughput characterization of aptamer-target binding and multiplexed assays for analytes in a complex biological sample. Upon aptamer binding to an analyte target, the binding event can be detected to characterize the presence and concentration of various analytes in the biological sample. However, because protein or other analyte concentrations can vary to a high degree within and/or between different biological samples, identifying a useful detection range for a multiplexed aptamer-based assay is difficult. Further, particular design constraints of aptamer molecules may result in challenges for downstream detection workflows.

### BRIEF DESCRIPTION

In one embodiment, the present disclosure provides a method of aptamer detection that includes contacting analytes of a sample with a plurality of aptamers under conditions that permit analyte-aptamer complexes to form, wherein different aptamers of the plurality of aptamers have specific affinity for respective different analytes of the analytes and detecting the analytes by detecting aptamers of the analyte-aptamer complexes. Detecting the individual aptamer includes contacting the individual aptamer with an exonuclease to deprotect a 3′ end of the individual aptamer to generate a deprotected 3′ end of the individual aptamer; modifying the deprotected 3′ end of the individual aptamer to generate a modified 3′end of the individual aptamer; capturing the individual aptamer using the modified 3′ end; and detecting the captured individual aptamer.

In one embodiment, the present disclosure provides a method of aptamer detection that includes contacting analytes of a sample with a plurality of aptamers under conditions that permit analyte-aptamer complexes to form, wherein different aptamers of the plurality of aptamers have specific affinity for respective different analytes of the analytes and detecting the analytes by detecting aptamers of the analyte-aptamer complexes. Detecting the individual aptamer includes modifying a 3′ end of the individual aptamer to generate a modified 3′end of the individual aptamer; hybridizing an oligonucleotide to the individual aptamer, wherein the oligonucleotide comprises a nonhybridizing 5′ region; extending an oligonucleotide 3′ end to generate an extended strand; and using the extended strand to generate a fragment of a sequencing library.

In one embodiment, the present disclosure provides an aptamer detection probe set. The probe set includes a plurality of different probe mixtures complementary to respective different aptamers of an aptamer panel, wherein an individual probe mixture of the plurality of different probe mixtures comprises a binding subset of probes coupled to an affinity tag; and a dummy subset of probes not coupled to the affinity tag, wherein each probe in the binding subset and the dummy subset of the individual probe mixture comprises a same binding region that is complementary to at least a portion of an individual aptamer of the aptamer panel and wherein each probe in the binding subset and the dummy subset of the individual probe mixture comprises a nonhybridizing region at a 3′ end.

In one embodiment, the present disclosure provides an aptamer detection probe set. The probe set includes a plurality of different probe mixtures complementary to respective different aptamers of an aptamer panel, wherein an individual probe mixture of the plurality of different probe mixtures comprises a binding subset of probes; and a dummy subset of probes comprising a modified 5′ end that cannot be ligated, wherein each probe in the binding subset and the dummy subset of the individual probe mixture comprises a same binding region that is complementary to at least a portion of an individual aptamer of the aptamer panel and wherein the binding subset of probes have an unmodified 5′ end that is capable of being ligated.

In one embodiment, the present disclosure provides a method of aptamer detection. The method includes contacting analytes of a sample with a plurality of aptamers under conditions that permit analyte-aptamer complexes to form, wherein different aptamers of the plurality of aptamers have specific affinity for respective different analytes of the analytes and detecting the analytes by detecting aptamers of the analyte-aptamer complexes. Detecting an individual aptamer of the plurality of aptamers includes contacting the individual aptamer with a single-stranded nucleic acid reporter probe to form an aptamer-reporter probe complex, the reporter probe comprising: an aptamer binding region that binds to the individual aptamer to form a first double-stranded region; a single-stranded region comprising a cleavage region and an identification sequence uniquely identifying for the individual aptamer or an associated analyte. Detecting the individual aptamer also includes extending the individual aptamer from a 3′ end to form a second double-stranded region comprising the identification sequence and using the single-stranded region as a template; separating the first double-stranded region from the second double-stranded region at the cleavage region; and sequencing the second double-stranded region to detect the identification sequence.

In one embodiment, the present disclosure provides an aptamer detection reporter probe set that includes a plurality of different single-stranded nucleic acid reporter probes complementary to respective different aptamers of an aptamer panel. A first single-stranded nucleic acid reporter probe of the plurality of different single-stranded nucleic acid reporter probes includes a first aptamer binding region that binds to a first individual aptamer to form a first double-stranded region; a first cleavage region; and a first identification sequence uniquely identifying for the first individual aptamer. A second single-stranded nucleic acid reporter probe of the plurality of different single-stranded nucleic acid reporter probes includes a second aptamer binding region that binds to a second individual aptamer to form a second double-stranded region; a second cleavage region; and a second identification sequence uniquely identifying for the second individual aptamer; wherein the first aptamer binding region and the second aptamer binding region have different nucleotide sequences relative to one another.

In one embodiment, the present disclosure provides a method of aptamer detection. The method includes contacting an individual aptamer with a reporter probe that hybridizes to the individual aptamer at an aptamer binding region to form a first double-stranded region of the reporter probe and wherein the reporter probe comprises a nonhybridizing region comprising a cleavage region and an identification sequence uniquely identifying for the individual aptamer or an associated analyte; extending the individual aptamer from a 3′ end to form a second double-stranded region using the non-hybridizing region as a template; separating the first double-stranded region from the second double-stranded region at the cleavage region; and detecting the identification sequence.

In one embodiment, the present disclosure provides a method of aptamer detection. The method includes contacting analytes of a sample with a plurality of aptamers under conditions that permit analyte-aptamer complexes to form, wherein different aptamers of the plurality of aptamers have specific affinity for respective different analytes of the analytes; and detecting the analytes by detecting aptamers of the analyte-aptamer complexes. Detecting an individual aptamer of the plurality of aptamers includes contacting the individual aptamer with an exonuclease to deprotect a 3′ end of the individual aptamer to generate a deprotected 3′ end of the individual aptamer; extending the deprotected 3′ end of the individual aptamer using a polymerase to generate an extended 3′end of the individual aptamer; hybridizing the individual aptamer to a reporter probe, wherein the reporter probe has a protected 3′ end that is not deprotected by the exonuclease or extended by the polymerase; capturing the individual aptamer using the modified 3′ end; and detecting the captured individual aptamer using the reporter probe.

### BRIEF DESCRIPTION OF THE DRAWINGS

These and other features, aspects, and advantages of the disclosed embodiments will become better understood when the following detailed description is read with reference to the accompanying drawings in which like characters represent like parts throughout the drawings, wherein:

FIG. 1 is a flow diagram of an aptamer detection technique that may be used in conjunction with aptamers and/or reporters that bind to aptamers, according to an embodiment of the present disclosure;

FIG. 2 is a schematic illustration of an aptamer modification technique using splint ligation, according to an embodiment of the present disclosure;

FIG. 3 is a schematic illustration of an aptamer bi-molecular assay, according to an embodiment of the present disclosure;

FIG. 4 is a schematic illustration of an aptamer modification technique using a terminal transferase and a hybridized poly-A, according to an embodiment of the present disclosure;

FIG. 5 is a schematic illustration of an aptamer modification technique using a hybridized degenerate probe, according to an embodiment of the present disclosure;

FIG. 6 is a schematic illustration of an aptamer modification technique using an oligo-linked nucleotide (oNTP), according to an embodiment of the present disclosure;

FIG. 7 is a schematic illustration of an aptamer modification technique using a hybridized reporter probe, according to an embodiment of the present disclosure;

FIG. 8 is a schematic illustration of an aptamer modification technique using a hybridized reporter probe, according to an embodiment of the present disclosure;

FIG. 9 is a schematic illustration of an aptamer modification technique using a hybridized reporter probe, according to an embodiment of the present disclosure;

FIG. 10 is a schematic illustration of an aptamer modification with an enzymatically-cleavable protecting group, according to an embodiment of the present disclosure;

FIG. 11 shows cleavage of the enzymatically-cleavable protecting group, according to an embodiment of the present disclosure;

FIG. 12 is a schematic illustration of an aptamer modification technique using an enzymatically-cleavable protecting group and a polymerase, according to an embodiment of the present disclosure;

FIG. 13 is a schematic illustration of an aptamer modification with an enzymatically-cleavable protecting group and a ligase, according to an embodiment of the present disclosure;

FIG. 14 is a schematic illustration of an aptamer detection workflow;

FIG. 15 shows an example aptamer set and reporter probe set, according to an embodiment;

FIG. 16 shows example aptamer-reporter probe complexes formed by contacting reporter probes with the aptamer set of FIG. 15, according to an embodiment;

FIG. 17 shows example 3′ end deprotection of the aptamer-reporter probe complexes of FIG. 16, according to an embodiment;

FIG. 18 shows an extended 3′ end after deprotection as shown in FIG. 17, according to an embodiment;

FIG. 19 shows separation of double-stranded regions after extension as shown in FIG. 18, according to an embodiment;

FIG. 20 shows double-stranded regions remaining after digestion of unbound reporter probes, according to an embodiment;

FIG. 21 shows sequencing adapter incorporation for separated double-stranded regions include the identification sequence, according to an embodiment;

FIG. 22A shows data from a model system demonstrating that DNA without an inverted dT can be extended by a polymerase with biotinylated nucleotides;

FIG. 22B shows data from a model system demonstrating that DNA with an inverted dT can be deprotected by ExoIII and extended by a polymerase with nucleotides and is inert to extension without deprotection;

FIG. 23A shows data from a model system demonstrating that DNA with an inverted dT can be deprotected by ExoIII and extended with nucleotides and is inert to extension without deprotection;

FIG. 23A shows data from a model system demonstrating that DNA with an inverted dT can be deprotected by ExoIII and extended by a polymerase with multiple biotinylated nucleotides and is inert to extension without deprotection;

FIG. 23B shows data from a model system demonstrating that DNA with an inverted dT can be deprotected by ExoIII and extended by a polymerase with a biotinylated nucleotide and is inert to extension without deprotection;

FIG. 24 is an example reporter with an identification sequence or ID at a 3′ end, according to an embodiment;

FIG. 25 is an example reporter with an identification sequence or ID at a 5′ end, according to an embodiment;

FIG. 26 is an example deprotect and extend workflow according to an embodiment;

FIG. 27 shows that 3′ inverted dT can be removed and that, after removal, extension with biotinylated nucleotides occurs for a group of aptamers; and

28 is a block diagram of a sequencing device configured to acquire sequencing data, according to an embodiment.

### DETAILED DESCRIPTION

The following discussion is presented to enable any person skilled in the art to make and use the technology disclosed, and is provided in the context of a particular application and its requirements. Various modifications to the disclosed implementations will be readily apparent to those skilled in the art, and the general principles defined herein may be applied to other implementations and applications without departing from the spirit and scope of the technology disclosed. Thus, the technology disclosed is not intended to be limited to the implementations shown, but is to be accorded the widest scope consistent with the principles and features disclosed herein.

Aptamers are short single stranded nucleic acid molecules (ssDNA or ssRNA) that can bind to their specific target molecules with high affinity. Accordingly, aptamers can be used for multiomic applications, such as proteome characterization of a sample in a high-throughput manner. Quantification and detection of aptamers can be used to indirectly quantify/measure/detect the target protein for the aptamer. There are numerous assays that utilize aptamers for protein detection. Certain aptamers may have modified nucleotides to enhance affinities to target proteins through slow off rates. To facilitate detection by next generation sequencing (NGS) techniques, aptamers retained from assays, or proxy molecules, are converted into sequencing (NGS) libraries in a quantitative and reproducible manner. Detection methods may directly sequence a copy of the aptamer sequence to identify it or translate the aptamer sequence into a barcode that can be sequenced to indirectly identify the aptamer and, therefore, a presence and/or concentration of the aptamer target in the sample of interest.

Aptamers may include protecting groups for nuclease resistance. The 3′ end can contain inverted dT to provide resistance against naturally occurring nucleases in biological samples or during storage of reagents. See Ni, S.; Yao, H.; Wang, L.; Lu, J.; Jiang, F.; Lu, A.; Zhang, G. Chemical Modifications of Nucleic Acid Aptamers for Therapeutic Purposes. Int. J. Mol. Sci. 2017, 18, 1683. The disclosed embodiments show how removal or replacement of the 3′ dT can afford multiple opportunities to proceed into a library preparation for preparing sequencing libraries from aptamers. The disclosed embodiments provide solutions to generate sequencing libraries (e.g., NGS libraries) from aptamers via modifications to the aptamer end structure or to downstream retention steps. In an embodiment, the modification may include deprotection of 3′ aptamer groups before addition of library prep adaptors or elements that aid in further conversion into libraries. Aptamers are typically protected from nuclease degradation by modifying groups on 3′ end. In an embodiment, this protection is removed after the aptamer-based assay to enable library prep steps to proceed.

As used herein, an aptamer may refer to a non-naturally occurring nucleic acid that has specific binding affinity for a target molecule. The binding of the aptamer to the target molecule can result in catalytically changing the target molecule, reacting with the target molecule in a way that modifies or alters the target molecule or the functional activity of the target molecule, covalently attaching to the target molecule (as in a suicide inhibitor), and facilitating the reaction between the target molecule and another molecule. In one embodiment, the target molecule is a three dimensional chemical structure, other than a polynucleotide, that binds to the aptamer through a mechanism which is predominantly independent of Watson/Crick base pairing or triple helix binding. In an embodiment, the aptamer is not a nucleic acid having the known physiological function of being bound by the target molecule.

Aptamers include nucleic acids that are identified from a candidate mixture of nucleic acids. A specific binding affinity of an aptamer for its target may refer to aptamer binding to its target generally with a much higher degree of affinity than it binds to other, non-target, components in a mixture or sample. Different aptamers may have either the same number or a different number of nucleotides. Aptamers may be DNA or RNA and may be single stranded, double stranded, or contain double stranded regions. The aptamers discussed herein can be used in any diagnostic, imaging, high throughput screening or target validation techniques or procedures or assays for which aptamers, oligonucleotides, antibodies and ligands, without limitation can be used. Aptamers as disclosed herein may be used in aptamer-based assays, such as those disclosed in U.S. Pat. Nos. 7,855,054 and 7,964,356 and U.S. Publication Nos. US/2011/0136099 and US/2012/0115752.

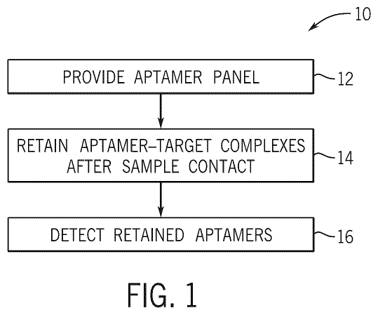

FIG. 1 shows an example flow diagram 10 for aptamer detection via an aptamer-based assay. In one example, a panel of aptamers to different target molecules is provided (block 12), e.g., provided attached to a solid support. The attachment of the aptamers to the solid support is accomplished by contacting a first solid support with the aptamer/s and allowing the releasable first tag included on the aptamer to associate, either directly or indirectly, with an appropriate first capture agent that is attached to or part of the first solid support. A test sample is then prepared and contacted with the immobilized aptamers that have a specific affinity for their respective target molecules, which may or may not be present in the sample. If the test sample contains the target molecule(s), an aptamer-target affinity complex will form in the mixture with the test sample. Aptamers that form complexes with analytes (e.g., targets) are retained (block 14) or separated from other components of the assay. In an embodiment, the retention is accomplished using a Catch-1 and Catch-2 partition as generally discussed herein. See Kraemer S, Vaught J D, Bock C, Gold L, Katilius E, et al. (2011) From SOMAmer-Based Biomarker Discovery to Diagnostic and Clinical Applications: A SOMAmer-Based, Streamlined Multiplex Proteomic Assay. PLOS ONE 6 (10): e26332.

In addition to analyte-aptamer complexes, uncomplexed aptamer will also be attached to the first solid support. The aptamer-target affinity complex and uncomplexed aptamer that has associated with the probe on the solid support is then partitioned from the remainder of the mixture, thereby removing free target and all other uncomplexed matter in the test sample (sample matrix); i.e., components of the mixture not associated with the first solid support. This partitioning step is referred to herein as the Catch-1 partition (see definition below). Following partitioning the aptamer-target affinity complex, along with any uncomplexed aptamer, is released from the first solid support using a method appropriate to the particular releasable first tag being employed.

In one embodiment, aptamer-target affinity complexes bound to the solid support are treated with an agent that introduces a second tag to the target molecule component of the aptamer-target affinity complexes. In one embodiment, the target is a protein or a peptide, and the target is biotinylated by treating it with NHS-PEO4-biotin. The second tag introduced to the target molecule may be the same as or different from the aptamer capture tag. If the second tag is the same as the first tag, or the aptamer capture tag, free capture sites on the first solid support may be blocked prior to the initiation of this tagging step. In this exemplary embodiment, the first solid support is washed with free biotin prior to the initiation of target tagging. Tagging methods, and in particular, tagging of targets such as peptides and proteins are described in U.S. Pat. No. 7,855,054.

Partitioning is completed by releasing of uncomplexed aptamers and aptamer-analyte complexes from the first solid support. In one embodiment, the first releasable tag is a photocleavable moiety that is cleaved by irradiation with a UV lamp under conditions that cleave ≥90% of the first releasable tag. In other embodiments, the release is accomplished by the method appropriate for the selected releasable moiety in the first releasable tag. Aptamer-target affinity complexes may be eluted and collected for further use in the assay or may be contacted to another solid support to conduct the remaining steps of the assay.

In one embodiment, a second partition is performed (referred to herein as the Catch-2 partition, see definition below) to remove free aptamer. As described above, in one embodiment, a second tag used in the Catch-2 partition may be added to the target while the aptamer-target affinity complex is still in contact with the solid support used in the Catch-0 capture. In other embodiments, the second tag may be added to the target at another point in the assay prior to initiation of Catch-2 partitioning. The mixture is contacted with a solid support, the solid support having a capture element (second) adhered to its surface which is capable of binding to the target capture tag (second tag), preferably with high affinity and specificity. In one embodiment, the solid support is magnetic beads (such as DynaBeads MyOne Streptavidin C1) contained within a well of a microtiter plate and the capture element (second capture element) is streptavidin. The magnetic beads provide a convenient method for the separation of partitioned components of the mixture. Aptamer-target affinity complexes contained in the mixture are thereby bound to the solid support through the binding interaction of the target (second) capture tag and the second capture element on the second solid support. The aptamer-target affinity complex is then partitioned from the remainder of the mixture, e.g. by washing the support with buffered solutions, including buffers comprising organic solvents including, but not limited to glycerol.

Aptamers are then eluted from aptamer-target complexes with buffers comprising chaotropic salts from the group including, but not limited to sodium perchlorate, lithium chloride, sodium chloride and magnesium chloride. Aptamers retained on Catch-2 beads by virtue of aptamer/aptamer interaction are not eluted by this treatment.

In another embodiment, the aptamer released from the Catch-2 partition is detected and optionally quantified by detection methods (block 16) as discussed herein, such as via next generation sequencing techniques. For example, detection may occur via amplification and/or sequencing of probes that bind to the eluted aptamers. In certain embodiments, the detection includes detection results that provide relative and/or estimated absolute concentrations of detected aptamers. The detection results may include a notification or output of a positive or negative detection result or a relative concentration or estimated concentration for a particular aptamer ID or a particular target of the aptamer.

The disclosed embodiments relate to aptamer detection techniques that may be used in conjunction with the flow diagram 10. In certain embodiments, the techniques include direct modification of aptamers. The modifications facilitate adapter incorporation for generating a sequencing library that is sequenced as part of aptamer detection. In embodiments, the techniques include incorporation of or modification of reporter probes that hybridize to aptamers, whereby the reporter probes are used to generate a sequencing library and are sequenced as part of aptamer detection.

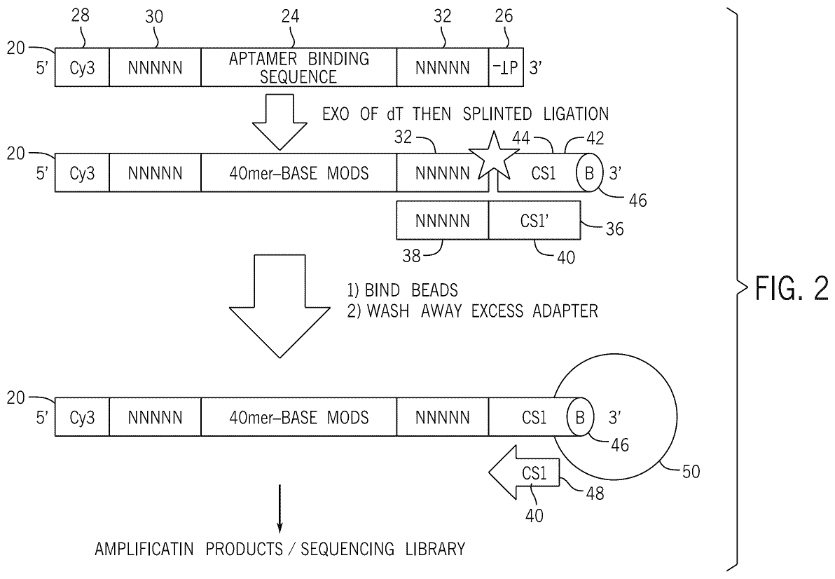

FIG. 2 shows an example workflow for aptamer modification of the illustrated aptamer 20. As show, the aptamer 20 may be converted into a sequencing library using a direct approach. The illustrated aptamer 20 includes an aptamer binding sequence 24 that functions to bind to a target of interest and that is retained for detection in the aptamer-based assay as a result of target binding. The aptamer 20 includes an inverted dT 26 at 3′ end and in embodiments, may include a label 28 at a 5′ end. One or more adjacent sequences 30, 32 are positioned at a 5′ position and/or a 3′ position of the aptamer binding sequence 24 and, in embodiments, do not form part of the aptamer-target direct binding. For example, these adjacent sequences 30, 32 may be sequences that are used during SELEX process and appended to the aptamer 20. In an embodiment, the adjacent sequences 30, 32 flank the aptamer binding sequence 24. In an embodiment, the adjacent sequences 30, 32 have respective nucleotide sequences that are fixed sequences uniquely associated with an individual aptamer binding sequence 24 and that are distinguishable from each other as well as the adjacent sequences 30, 32 of different aptamers 20. Thus, for an aptamer panel of N different aptamers 20 having respective N different aptamer binding sequences 24, there may also be N different adjacent sequences 30 and/or N different adjacent sequences 32. In an embodiment, only 3′ adjacent sequence 32 is present. The adjacent sequences 30 and 32 may also be shared between different aptamer binding sequences.

The adjacent sequences 30, 32 are shown as being Nmers of 5 nucleotides. However, other lengths are also contemplated. In an embodiment, one or both of the adjacent sequences 30, 32 may be 4-25 nucleotides in length. In an embodiment, one or both of the adjacent sequences 30, 32 may be directly adjacent to the aptamer binding region 24 such that no intervening nucleotides are present between these sequences. As discussed below, because of direct ligation of the adjacent sequence 32 to a splinted oligonucleotide, the adjacent sequence 32 may be directly adjacent to the inverted dT 26 at 3′ end.

In a first step of the illustrated workflow, an exonuclease is provided as well as a first oligonucleotide 36 and contacted with the aptamer 20. The first oligonucleotide includes a complementary region 38 to the adjacent sequence 32 positioned at the 3′ end as well as an adapter sequence 40 (shown as CS1′ by way of example). The first oligonucleotide 36 is provided as a single-stranded oligonucleotide that, upon contact with the aptamer 20 having the complementary adjacent sequence 32, becomes at least partially double-stranded. The adapter sequence 40 is positioned 5′ of the complementary region 38 and does not bind directly to the aptamer 20 but is coupled via the complementary binding of the complementary region 38. The adapter sequence 40 provides a common adaptor for PCR later in the process.

The first oligonucleotide 36 and the exonuclease may be provided sequentially or together. In an embodiment, the exonuclease is a single-stranded or double-stranded exonuclease that is provided before the first oligonucleotide 36 such that the deprotection or inverted dT 26 removal is conducted on a single-stranded region of the aptamer 20. The exonuclease may be a single-stranded DNA 3′ to 5′ exonuclease, such as Exo I or may be ExoIII. Exonuclease removal of 3′ inverted dT group from 3′ end of the aptamer 20 allows the ligation of a second oligonucleotide 42 including a complementary adapter sequence 44 (shown as CS1) to bind to its complement via a splinted ligation process. The 3′ end of the second oligonucleotide 42 is biotinylated 46. The second oligonucleotide 42 is provided as a single-stranded oligonucleotide that, upon binding to the adapter sequence 40, becomes part of a double-stranded structure. The binding permits ligation of 3′ end of the deprotected aptamer 20 via binding of 3′ terminal nucleotide of the adjacent sequence 32 to 5′ end of the second oligonucleotide 42 via a ligase. Thus, the first oligonucleotide 36 includes a first portion, e.g., the complementary region 38, that binds to the aptamer 20, and a second portion, e.g., the adapter sequence 40, that binds to the second oligonucleotide 42.

After splint ligation to ligate the second oligonucleotide 42 to the aptamer 20, the aptamer 20 includes the complementary adapter sequence 44 at 3′ end and is thus partially adapterized. A solid phase may be to be used (i.e. beads 50 that bind to the biotin) to capture individual aptamers 20 and to wash away any unbound first oligonucleotides 36 not bound to aptamers 20. The first oligonucleotide 36 may be separated from the modified (adapterized) aptamer 20. In an embodiment, the oligonucleotide 36 may be removed before or after bead-bound washing. This may occur using a denaturation and annealing step. Following washing, extension from a primer 48 that binds to the complementary adapter sequence 44 can create a double stranded DNA copy of the aptamer 20. When a copy of the aptamer 20 is attempted, a polymerase that can tolerate modified bases may be used. This copied strand can be used to generate amplification products for DNA library preparation through available single stranded library preparation methods, which may include additional ligation steps and/or use of adapter-overhang primers for amplification using the copied strand as a template. Use of a universal or fixed adapter sequence 40 that does not vary between different adjacent sequences 32 associated with different aptamer binding sequences 24 permits extension of all aptamers 20 of a panel using a common primer 48. That is, while different first oligonucleotides 36 used with an aptamer panel may have different complementary regions 38 having respective different sequences, all adapter sequences 40 may be the same between different first oligonucleotides. Further, all second oligonucleotides 42 may have a same complementary adapter sequence 44.

Thus, for a given aptamer panel, multiple different first oligonucleotides 36 and same second nucleotides 42 may form a set of probes used in conjunction with aptamer detection.

Embodiments of the present disclosure may be used in conjunction with dynamic range compression techniques. For example, different aptamers retained in an aptamer-based assay may be present in different ranges depending on the overall abundance of their target. The disclosed oligonucleotides that are either ligated to or annealed to the aptamers 20 may include an affinity tag such that the bound aptamers are retained via affinity tag binding. By providing a mixture of dummy oligonucleotides without the affinity tag, the overall retention of aptamers may be affected. That is, dummy oligonucleotides will bind to the aptamers 20, rendering them unavailable for binding to an oligonucleotide with the affinity tag. Those aptamers 20 that do not complex with a tagged oligonucleotide are not retained by the affinity tag capture molecule (e.g., the bead 50, an affinity tag capture molecule linked to a substrate), and can be removed during a wash step that retains the beads 50 (e.g., via magnetic-based techniques). Thus, abundant aptamers 20 can be decreased in the mixture by selecting more dummy oligonucleotides while low concentration aptamers 20 can be relatively increased in comparison to other aptamers 20 in the mixture by not using any dummy oligonucleotides. In one example, the affinity tag (e.g., the biotin 46) is present on the first oligonucleotide 36 rather than the second oligonucleotide 42. Because each individual oligonucleotide is specific for a particular aptamer 20, an appropriate ratio of dummy first oligonucleotides (containing no affinity tag) and reporter first oligonucleotides (with the affinity tag) can be selected to decrease retention of an individual aptamer 20 of interest by changing a ration of dummy: reporter of the first oligonucleotides including the complementary region 38 targeting that individual aptamer 20.

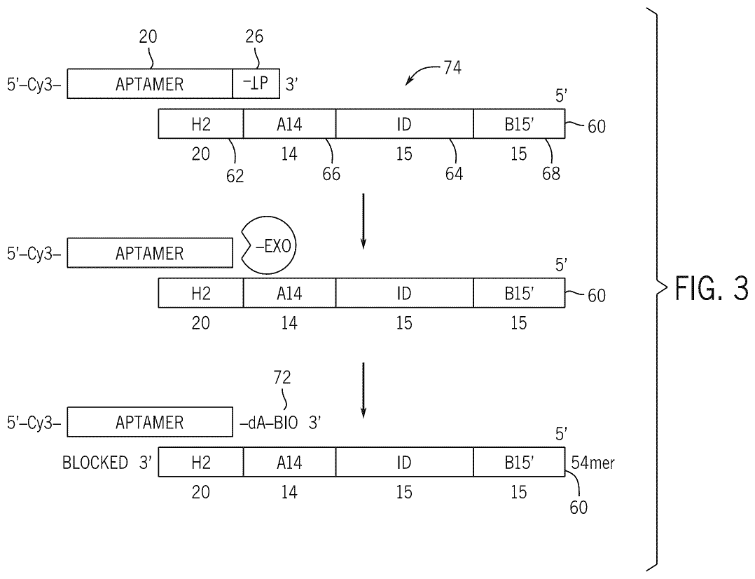

FIG. 3 shows an example of an indirect detection workflow using a reporter probe 60. The illustrated workflow shows a bi-molecular arrangement in which the aptamer 20 hybridizes to a reporter probe 60 to form a bi-molecular complex that may be retained via an affinity tag. The workflow includes a step of contacting the aptamer 20 with the reporter probe 60. Hybridization of the reporter probe 60 to the aptamer 20 is mediated through a complementary binding region 60 (denoted as H2). The reporter probe 60 includes an identification sequence 64 as well as primer sequences 66, 68 (shown by way of example as A14 and B15′ by way of example) for subsequent PCR. In this approach, the hybridized reporter probe 60 is used as a template to extend the aptamer 20. In embodiments, the template portion of the primer sequence 66 may form a mismatch with the inverted dT 26. If the available polymerase is a polymerase with 3′ to 5′ exonuclease activity, the polymerase, via its exonuclease activity, may remove 3′ inverted dT group 26. The polymerase may be T7 DNA polymerase or any other polymerase with ‘3-5’ activity. The polymerase may be used to extend the aptamer 20 (using the bound reporter probe 60) with a biotinylated nucleotide 72 and using the aptamer 20 as the template. Alternatively, an exonuclease (double or single stranded such as ExoI or ExoIII) may be used to deprotect before contact with the reporter probe 60, and the polymerase may be provided as a separate enzyme to the reaction mixture or the polymerase (e.g., taq) and exonuclease could be included as a cocktail or kit.

This biotinylated nucleotide 72 can be used as a handle for a solid support to apply stringent washes and remove the excess reporter probes 60 from the system. By adding a biotin group to the 3′ end of the aptamer 20 allows more of the aptamer 20 to be used for the binding to the reporter probe complementary binding region 62, and therefore have higher Tm, and increased stringency where necessary. The retained reporter probes 60 can be amplified and sequenced.

The reporter probe 60 includes the complementary binding region 62, which may be selected to be sufficient in length for selective binding to the target aptamer 20. In the depicted embodiment, the complementary binding region 62 is 20 nucleotides long. In an embodiment, the complementary binding region 62 may be 5-50 nucleotides in length. The reporter probe includes a nonhybridizing region 74 that extends away from the complementary binding region 62 and that does not hybridize to the aptamer 20. Thus, the sequence of the nonhybridizing region 74 can be selected to avoid substantial complementarity with a sequence of the aptamer 20. The nonhybridizing region 74 can be used for detection as a proxy for the aptamer 20. Accordingly, the nonhybridizing region 74 the identification sequence 64 is unique to the individual aptamer 20. Thus, different aptamers 20 are associated with respective different identification sequences 64 that are all different from one another and are uniquely identifying. In an embodiment, uniquely identification sequences 64 are uniquely identifying while accounting for barcode errors (e.g., a 1-2 nucleotide sequence error) during sequencing. Further, the identification sequence 64 may be designed such that the identification sequence 64 is different from the aptamer sequence.

To facilitate detection, the nonhybridizing region 74 can include a first adapter region 66 and a second adapter region 68 that flank the identification sequence 64 to facilitate amplification of the nonhybridizing region 74 using universal or conserved primers to generate amplification products as part of preparation of a sequencing library for sequencing. The adapter regions 66, 68 may be part of adapter sequences of sequencing libraries. As illustrated, the first primer region 66 is 14 nucleotides in length and the second adapter region 68 is 15 nucleotides in length. The identification sequence 64 is 15 nucleotides in length. Thus, the total length of the reporter probe 60, as illustrated, is 54 nucleotides by way of example.

The identification sequence as provided herein, e.g., identification sequence 64, can include one or more nucleotide sequences that can be used to identify one or more specific aptamers 20. The identification sequence can be an artificial sequence. The identification sequence can comprise at least about 1, 2, 3, 4, 5, 6, 7, 8, 9, 10, 11, 12, 13, 14, 15, 16, 17, 18, 19, 20 or more consecutive nucleotides. In some embodiments, the identification sequence comprises at least about 10, 20, 30, 40, 50, 60, 70 80, 90, 100 or more consecutive nucleotides.

The adapters as provided herein may be 5-50 nucleotides in length, e.g., 5-50 consecutive nucleotides. Thus, a total reporter probe length may be 20-200 nucleotides or more.

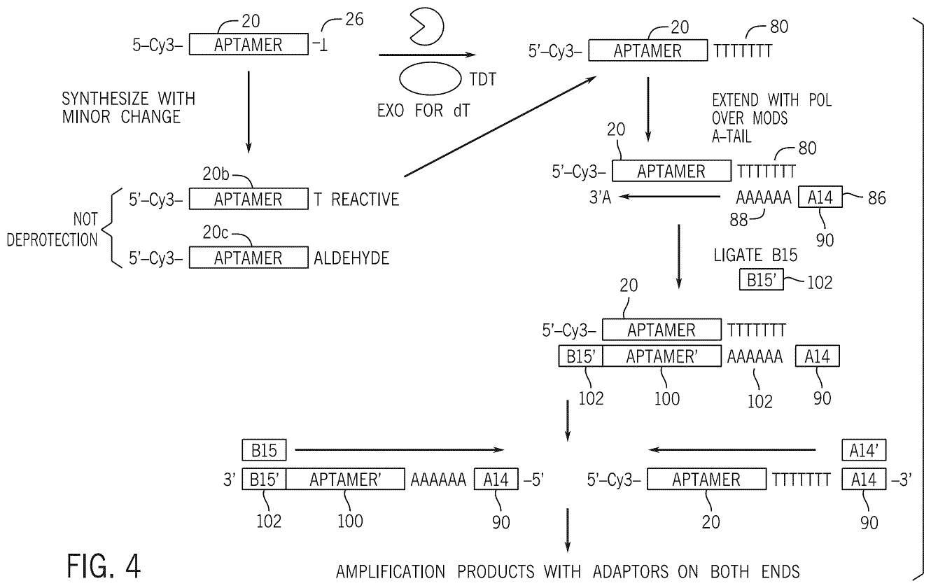

FIG. 4 is an example workflow for different implementations of the aptamer 20 in which a poly-T region is incorporated in a template-independent manner. An aptamer 20a including 3′ inverted dT may, as an initiating step, have 3′ inverted dT group is removed with an exonuclease (e.g., a single-stranded exonuclease). In the same reaction (or subsequent reaction), terminal transferase (TdT) is used to add bases 80 (poly T) to the 3′ end of the aptamer 20a in a template-independent reaction. The polyT 80 can be used as a primer site for a primer 86 that includes a poly A region 88 and a PCR primer region 90 (shown by way of example as A14). Extension of the primer 86 using a polymerase effectively copies the single strand aptamer 20 into a copied strand 100 and appends an adaptor including the PCR primer region 90 to 5′ end of the copied strand 100. Following extension using a modification-tolerant polymerase, a second adaptor is required for PCR and sequencing. This can be added through A-tailing and ligation of a second adaptor 102 at the end of the duplex. The copied aptamers 20 can be amplified and sequenced as illustrated.

Over multiple amplification cycles, the population of the strands including both 5′ and 3′ adapter sequences will become predominant, because the initial aptamer 20, and strands copied from it, with only the first adapter 90 will not be picked up by primers targeting the second adapter 102. The generated amplification products with adaptors on both ends may form all or part of a sequencing library that is used in a sequencing reaction to generate sequencing data

Other implementations of the aptamer 20 may enter the workflow at different stages. For example, an aptamer 20b that includes a conventional or unprotected 3′ end may not undergo exonuclease treatment. An aptamer 20c modified with an aldehyde group at a 3′ terminus may also undergo excision based on polymerase proofreading activity or may be used in a chemical biotinylation reaction. The aldehyde group may serve to protect 3′ end from nucleases, and a variety of polymerases with aldehyde lesion removal activity may be available.

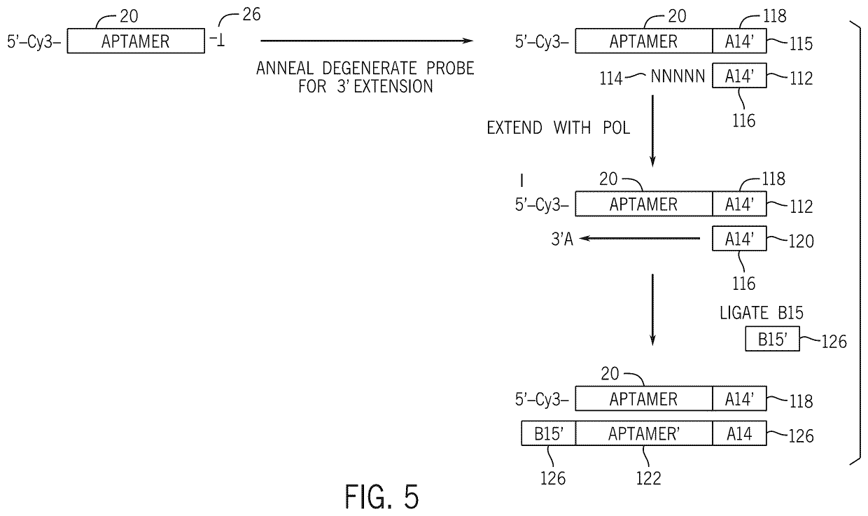

FIG. 5 shows a reaction in which a binding first oligonucleotide 112 is composed of degenerate sequences 114 (NNNNN). This mix of degenerate sequences 114 can be synthesized in a single reaction and provided to an aptamer panel including individual aptamers 20. That is, the degenerate sequences may be generated in a less complex manner relative to the example in FIG. 2 in which each oligonucleotide complementary to the aptamer 20 is synthesized separately. The first oligonucleotides 112 all include a common or shared adapter sequence 116 and different degenerate sequences 114. Any first oligonucleotides with a complementary binding site on the aptamer 20 bind to provide an available binding site for a second oligonucleotide 115. The exonuclease deprotection of the inverted dT end 26 and polymerase extension results in ligation of the second oligonucleotide 115 that includes a common 3′ adaptor 118 directly to the 3′ end of the aptamer 20. In this manner, 3′ end of the aptamer 20 is adapterized. While the use of degenerate sequences 114 may result in binding to locations of the aptamer 20 5′ of the desired 3′ end binding site, such binding will not provide the overhanging 5′ end of the first oligonucleotides 112 that permits the splint ligation.

The 5′ end of the aptamer 20 can be adapterized as shown in FIG. 4 via amplification and ligation. Polymerase extension from the first oligonucleotide 112 or a primer 120 complementary to the adapter 118 creates a copied strand 122. Then a ligation reaction can be performed to add a 3′ adaptor 126 to the copied strand 122 as described in FIG. 4. Subsequent amplification with primers complementary to the end adaptors, e.g., as in FIG. 4, can be used to generate amplification products with adaptors on both ends. The amplification products may form all or part of a sequencing library that is used in a sequencing reaction to generate sequencing data.

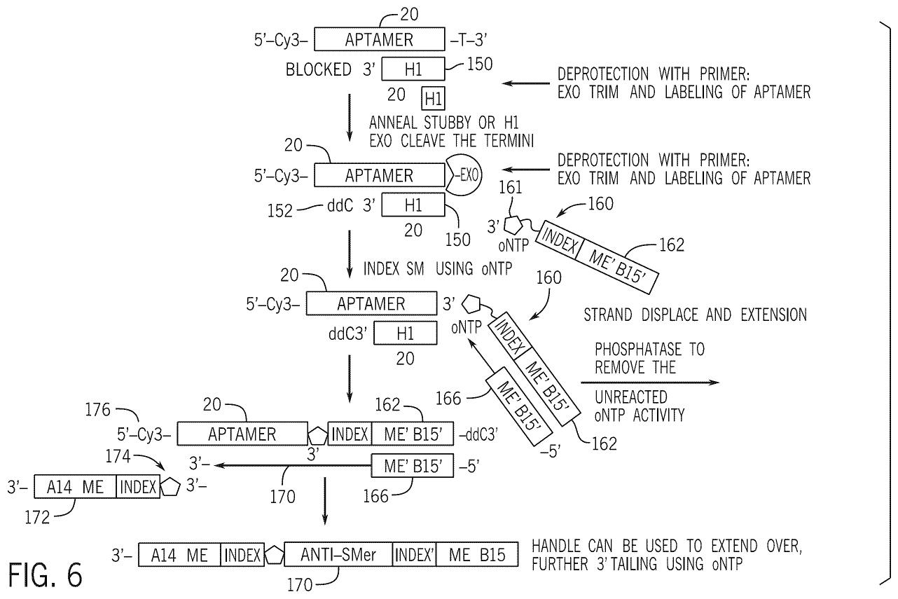

The presence of a Cy3 dye at the 5′ end of the aptamer 20, which is used as part of aptamer-based assays, may make ligation of 3′ adaptor to the copied stand (FIGS. 4-5) inefficient. To overcome this, an oligo linked nucleotide library prep approach, as shown in FIG. 6, may be used. Oligo-linked oligonucleotides (ONTPs) as discussed in WO2022251510A2, incorporated by reference herein in its entirety for all purposes, may be used. oNTPs harness the ability of polymerases to catalyze the incorporation of nucleotides that are coupled to oligonucleotide adapters (or other functional sequences). Thus, in embodiments, desired sequences can be added to 3′ ends of nucleic acids via a polymerase-mediated reaction rather than a ligation reaction, and the use of polymerase permits higher yield of the desired end product relative to ligation. As shown in FIG. 6, the disclosed oligo-modified nucleotide analogues include adapter sequences that are used to incorporate adapters during sequencing library preparations. Compared to ligase-mediated adapterization of nucleic acid samples, the direct incorporation via polymerase of modified nucleotides conjugated to sequencing adapters increases the efficiency of library preparation and simplifies user work flows. Implementations also facilitate asymmetric adapterization of libraries, e.g., with different 5′ and 3′ adapters to permit production of stranded libraries and paired end sequencing.

oNTPs contain oligos as modified linkages to the base group. It has been demonstrated that after incorporation of one of these oNTPs, an extension can occur from an adaptor linked to the oNTP base, which will cross the unusual ‘lesion’ in the DNA and extend across the original DNA template used for the oNTP addition. Thus, adaptors linked to bases may be added as sequences to 3′ ends of nucleic acids. In FIG. 6, The aptamer 20 may include the inverted dT protected end (removed by exonuclease) or a conventional end. In embodiments, the 3′ end of the aptamer 20 may be tailed (using TdT for dA tailing) to provide a reactive end. The oNTP 160 is added after hybridization of a probe 150 to a specific aptamer sequence, although degenerate approach could be used as in FIG. 5.

The oNTP nucleotide 161 is incorporated to the 3′ end of the aptamer 20 in a polymerase reaction. As shown, 3′ end 152 of the probe 150 may be blocked (using a ddNTP) such that the polymerase reaction to incorporate the oNTP at 3′ aptamer end does not incorporate the oNTP onto the probe 150. The oNTP adaptor 162, linked directly to the oNTP incorporated nucleotide 161, contains a sample index and sequencing primers (ME′ and B15′) by way of example. In an embodiment, the oNTP adaptor 162 has a blocked 3′ end to prevent polymerase incorporation during subsequent oNTP additions. Phosphatase can be used to treat any unincorporated oNTPs 160 such that excess oNTPS 160 are not incorporated during addition of a second, different oNTP at later stages.

Once incorporated, a strand displacing polymerase can be used to prime from B15′ (using primer 166) and cross over the oNTP base ‘lesion’ and across the aptamer 20 as shown. This effectively adds a 5′ adaptor onto the copied strand 170 that is the complement of the oNTP adaptor 162. The same approach can be repeated to add a second adaptor 172, via addition of a second oNTP 174 at 3′ end via polymerase incorporation. Polymerase incorporation may be more robust than ligation-based reactions and, therefore, may not be as affected by a presence of a label 176 at a 56′ end of the aptamer 20. The copied strand 170 is a complement of the aptamer 20 and has adaptor sequences appended to both ends. Amplification products of the copied strand can be used to generate a sequencing library to generate sequencing data.

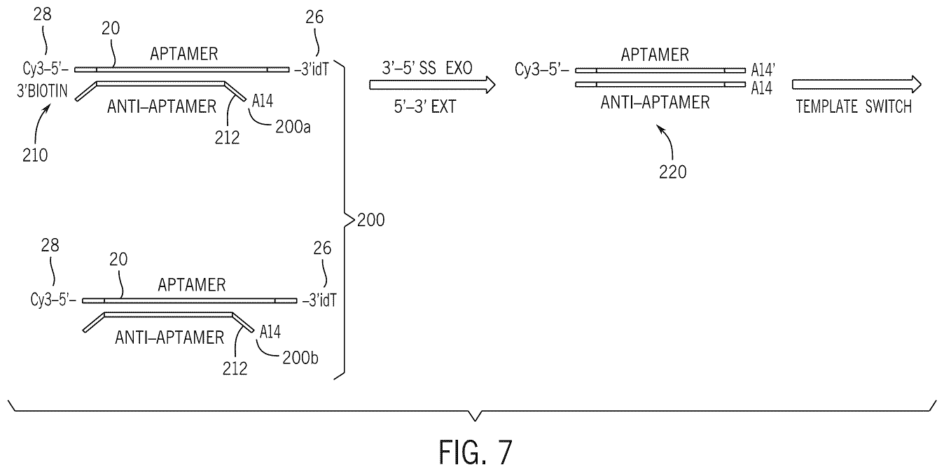

FIG. 7-9 show multiple methods for deprotecting 3′ and 5′ sequences for library prep. Aptamers with 5′ Cy3 are inert from library preparation at the 5′ end in the same way as the inverted dT at 3′ end. In the illustrated methods a complementary sequence is hybridized to the aptamer 20 (anti-aptamer). The anti-aptamer may be full or partial length. Aptamers 20 can be captured on the probes using an affinity tag, and their signal can be titrated down if required for dynamic range compression as generally discussed herein. Using a combination of exonuclease and polymerase (3-5 ssExo and 5-3 extension or 5-3 ssExo) the aptamer hybridization complexes can be ‘blunted’ with three different outcomes as shown. Selection of exonuclease/polymerase depends on idT and Cy3 bond type, i.e., phosphodiester, phosphorothioate or phosphoroamidite. Following blunting, the three different double stranded molecules can be used for either template switch (polymerase extension library prep) or direct ligation of both or single adaptors to the ends as shown.

In FIG. 7, the aptamer 20 is annealed to a probe 200 to create a partially double-stranded complex with overhangs on both sides of the aptamer 20. In addition, the probe 200 include nonhybridized portions corresponding to the 3′ end and 5′ end. The 3′ overhanging portions of the aptamer 20 and the probe 200 may be removed by a 3′ to 5′ single-stranded exonuclease. Excess probes 200 can be digested by the exonuclease as well.

Removal of 3′ end nonhybridized portion may be accomplished after affinity tag retention and washing so that the unhybridized 3′ portion including the affinity tag 210 is present to facilitate affinity-tag based techniques. The 3′ end of the probe includes an affinity tag 210 (e.g., a biotin tag). In an embodiment, the probe 200 may be provided as probe set that includes a mixture of a subset of dummy probes 200b and a subset of capture probes (e.g., biotin-containing probes) 200a. The probes 200 may be otherwise identical in sequence, but either including or not including the biotin tag to facilitate dynamic range compression. Higher abundancy aptamers 20 may be provided with different dummy: biotin probes 200 than lower abundancy aptamers 20. For example, higher abundancy aptamer 20 may have a higher proportion of dummy probes relative to lower abundancy aptamers 20. It should be understood that, in embodiments, the probes 200 may include only biotin-containing probes 200a.

After retention, and any dynamic range compression, 3′ to 5′ exonuclease and 5′ to 3′ extension may be performed. This step may remove any attached affinity tag binding structures, such as a bead or substrate. The 5′ to 3′ extension generates a blunt-ended double-stranded structure 220 using 5′ overhangs as templates. The resultant double-stranded structure 220 with an adaptor on one end may be provided to a template switch library preparation protocol.

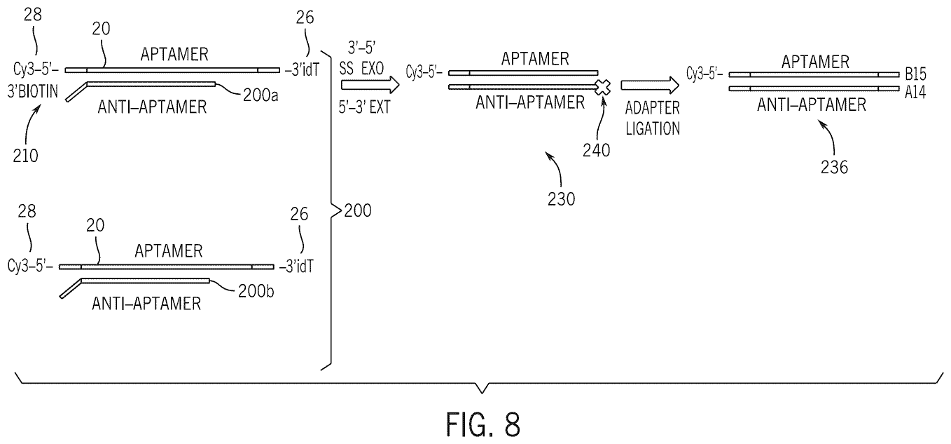

FIG. 8 shows an alternative arrangement that includes certain steps of FIG. 7 but in which the probe 200 does not include 5′ adaptor sequence 212 (see FIG. 7). Thus, performing the steps as in FIG. 7 yields a blunt end structure 230 without the adaptor sequence 212 at one end. In this example, the blunt-ended double-stranded structure 230 may be provided to an adaptor ligation technique to generate a double-stranded structure 236 that is partially adapterized or has a single-side adaptor.

As an alternative to providing a set of probes 200 with dummy 200b and biotin-containing 200a probes, the probes 200 may include a set with a modification 240 at a 5′ end that prevents adaptor ligation and, therefore, proceeding to subsequent library preparation steps. By adjusting the presence and/or ratio of dummy unligateable ends 240 in the population of probes 200, dynamic range compression may be achieved.

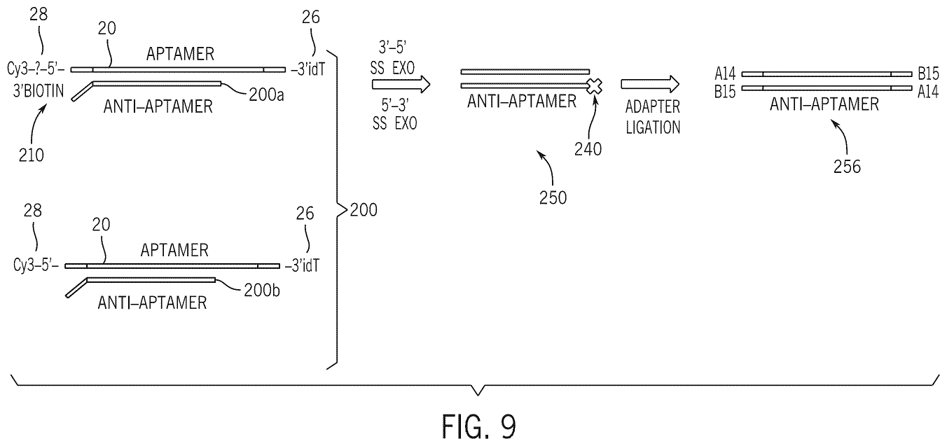

FIG. 9 shows an alternative arrangement that includes certain steps of FIG. 8. As in FIG. 8, performing the steps as in FIG. 7 yields a blunt end structure 230 without the adaptor sequence 212 at one end. However, by using a 3′ to 5′ exonuclease as well as a 5′ to 3′ exonuclease, all single-stranded regions are digested, leaving a blunt-ended double-stranded structure 250. Adaptors can be ligated to both blunt ends to generate an adapterized fragment 256.

As discussed with respect to FIG. 8, s an alternative to providing a set of probes 200 with dummy 200b and biotin-containing 200a probes, the probes 200 may include a set with a modification 240 at a 5′ end that prevents adaptor ligation and, therefore, proceeding to subsequent library preparation steps. By adjusting the presence and/or ratio of dummy unligateable ends 240 in the population of probes 200, dynamic range compression may be achieved.

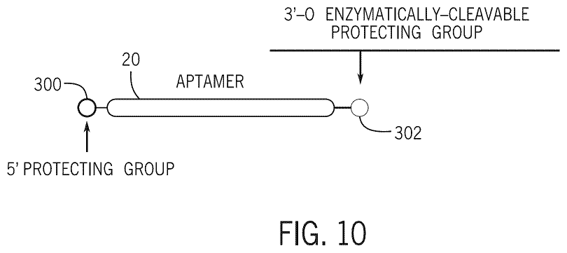

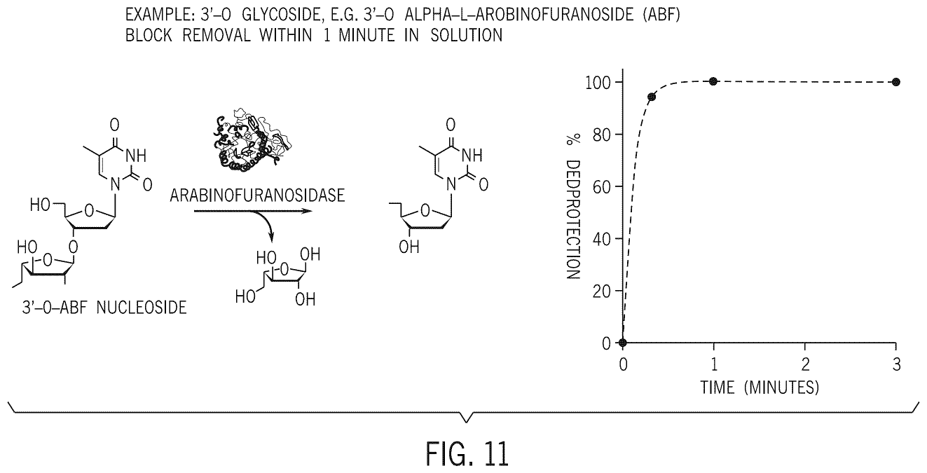

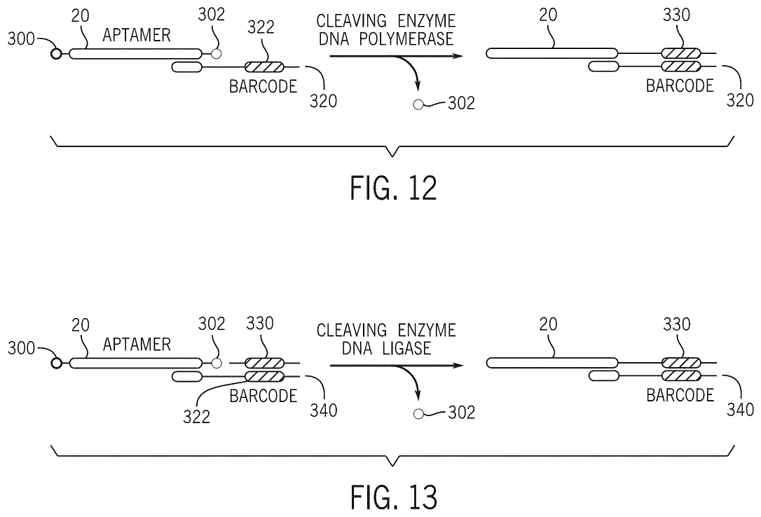

FIGS. 10-13 shows alternative approaches without an inverted dT for initial protection of the aptamer. As shown in FIG. 10, the aptamer 20 may include a 5′ protecting group 300 and a 3′ group that can be conveniently removed using efficient enzymes in a subsequent library prep. Here 3′ end is protected by a nucleotide containing 3′O 302 (e.g. 3′ glycoside). The 3′O ABF nucleoside is effectively deprotected by incubation with enzyme ABFase as shown in FIG. 11. For a library prep system, the aptamers 20 are be synthesized with 3′ protecting group 302. In a library prep workflow as shown in FIGS. 12-13, a reporter oligonucleotide 320, 340 is hybridized to the aptamer 20 and incubated with ABFase to deprotect 3′ end. The workflow of FIG. 12 shows a DNA polymerase extension example, while the workflow of FIG. 13 shows a DNA ligase example. Two options are shown for polymerase extension and ligation. Both methods would directly append an adaptor or barcode 322 (and its complement 330) to aptamer 20. This approach could be used as a substitute for any of the deprotecting methods herein.

In certain embodiments, aptamers in an aptamer-based assay can be detected via sequencing. However, aptamer sequences may not be optimized for clarity or distinguishability in sequencing results. That is, aptamers may be designed with sequences to optimize analyte interaction, and any changes to these sequences to improve sequencing results may negatively affect binding quality. Accordingly, it may be beneficial to detect proxy sequences, such as reporter probe identification sequences. For example, aptamer detection may involve indirect detection using a reporter probe that binds to (e.g., hybridizes to) an aptamer and that is detected, e.g., via sequencing. Thus, a reporter probe may include an identification sequence associated with a particular aptamer, such that a presence of the identification sequence in sequencing results is associated with a positive result for presence of the aptamer and/or the associated analyte. Use of a reporter probe may permit incorporation of additional detection functionality. A reporter probe may be designed to permit downstream incorporation of sequencing adapters and/or may have other features to enhance amplification or sequencing. Provided herein are reporter probes that include an integral cleavage region and that permit separation of an identification sequence from an aptamer binding region. In this manner, the identification sequence may be more efficiently detected. In certain embodiments, the separation may be accomplished without an initial amplification step.

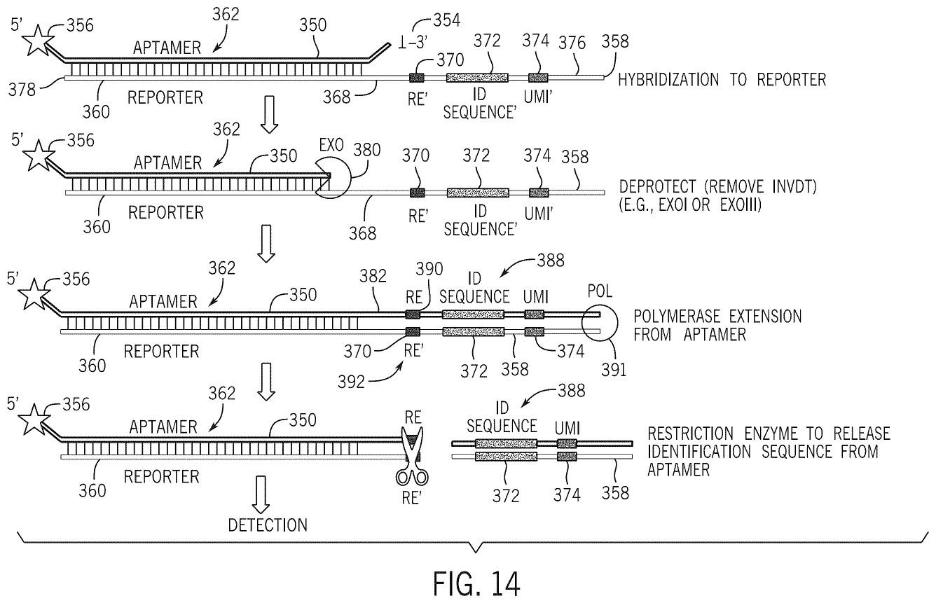

FIG. 14 shows an example workflow for reporter probe binding that may be used in conjunction with an aptamer-based assay. The aptamer-based assay can include analyte contacting steps and one or more separation steps to generate an eluate or solution including the illustrated aptamer 350. The aptamer 350 may include features that facilitate these assay steps, such as a label or dye (e.g., Cy5), shown here as positioned at an aptamer 5′ end 356 and a blocking or protective group positioned at an aptamer 3′ end 354. In an embodiment, the blocking or protecting group provides resistance to exonucleases and endonucleases. In an embodiment, 3′ cap is an inverted thymidine, such as a (3′-3′-linked) dT nucleotide. The illustrated aptamer 350 may be part of an assay eluate that is separated from other aptamers (not shown) that did not bind targets and that were not retained through separation steps.

Using the conserved sequence of the aptamer 350, a nucleic acid (e.g., DNA or RNA) reporter probe 358 can be designed. The reporter probe 358 includes an aptamer binding region 360 that is complementary to the aptamer 350. Thus, the sequence of the aptamer binding region 360 is based on the sequence of the aptamer 350. The reporter probe 358 also includes a nonhybridizing region 368 that extends away from the aptamer binding region 360 and that does not hybridize to the aptamer 350. Thus, the sequence of the nonhybridizing region 368 can be selected to avoid substantial complementarity with a sequence of the individual aptamer 350 or sequences of other aptamers 350. The nonhybridizing region 368 can be used for detection as a proxy for the aptamer 350. Accordingly, the nonhybridizing region 368 can include an identification sequence 372 that is unique to the individual aptamer 350. Thus, different aptamers 350 are associated with respective different identification sequences 372 that are all different from one another and are uniquely identifying. In an embodiment, uniquely identifying sequences are uniquely identifying while accounting for barcode errors (e.g., a 1-2 nucleotide sequence error) during sequencing. Further, the identification sequence 372 may be designed such that the identification sequence 372 is different from and/or nonoverlapping with the aptamer sequence.

The nonhybridizing region 368 may include an integral cleavage region 370. The cleavage region, while shown here in single-stranded form, when double-stranded after extension as disclosed herein, forms a double-stranded cleavage site. In an embodiment, the cleavage region forms all or part of a restriction site to facilitates cleavage using the appropriate restriction enzyme. The cleavage region 370 can be conserved across all aptamers 350 such that only a single restriction enzyme is required to cleave the nonhybridizing region. The cleavage site may be specific for double stranded or single stranded DNA cleavage.

In an embodiment, unique molecular identifiers (UMIs) 374 may be incorporated in the reporter probes 358. UMIs 374 are short sequences used to uniquely tag each molecule in a sample library to provide error correction and reduce sequencing bias. Accordingly, in an embodiment, different reporter probe molecules having a same aptamer binding region sequence may have different UMI sequences.

In the illustrated arrangement, the reporter probe 358 is shown with a 5′ end 376 and a 3′ end 378 such that the identification sequence 372 is 5′ of the cleavage region 370. The cleavage region 370 is positioned between the identification sequence 372 and the aptamer binding region 360. In an embodiment, the aptamer binding region 360 is 3′ of the cleavage region 370. In an embodiment, the aptamer binding region 360 extends to the 3′ end 378.

The reporter probe 358 may include linking sequences between the UMI 374, the identification sequence 372, the cleavage region 370, and/or the aptamer binding region 360. In an embodiment, an individual reporter probe 358 may have a universal or conserved sequence with the exception of the aptamer binding region 360 and/or the UMI 374.

The linking sequences may be conserved between different reporter probes 358 having respective different aptamer binding regions 360 and/or UMIs 374.

The reporter probe is complementary to the aptamer 350 such that contact with the aptamer 350 under hybridization conditions permits hybridization, e.g., binding. Thus, the bound reporter probe 358 creates a double-stranded region 362 and the single-stranded nonhybridizing region 368.

Subsequent to hybridization, 3′ end 354 of the aptamer 350 can be deprotected. In an embodiment, the deprotection may be via an exonuclease treatment, such as by ExoI or EXOIII. In an embodiment, the deprotection occurs via a timed incubation. In certain embodiments, the incubation is a timed incubation (e.g., 10 minutes) at 37° C., followed by a timed inactivation incubation (e.g., 10 minutes) at 70° C.

Deprotection of 3′ end 354 permits extension from 3′ end 354 using the nonhybridizing region 368 as the template. That is, the aptamer 350 acts as a primer for extension. Accordingly, deprotection may be followed by polymerase extension using a polymerase 391, such as taq polymerase using appropriate buffers and dNTP reagents. In cases where the exonuclease is heat-deactivated, a heat-tolerant polymerase may be provided together with the exonuclease in a single reagent mixture.

In another embodiment, the deprotection and extension may be accomplished using a polymerase with 3′ to 5′ exonuclease activity, such as taq plus. Other contemplated polymerases include NEB Q5, Pfu, Phi29, DNA pol, T4 Pol, Vent.

Extension from 3′ end of the aptamer 350 creates a complementary strand 382 to the nonhybridizing region 368 and a double-stranded structure. In an embodiment, the cleavage region 370 can create the restriction site 390 either alone or when the complementary strand is present. Further, the identification sequence 372 may be one or both of the sequence present on the nonhybridizing region 368 or the complementary sequence.

Cleavage at the restriction site 390 using a restriction enzyme 392 separates the first double-stranded region 362 from a second double-stranded region 388. Accordingly, the identification sequence 372 is no longer coupled to the associated aptamer 350. This permits separation of the separated double-stranded regions 362, 388 for downstream sequencing steps, in an embodiment. In an embodiment, the aptamer binding region 360 and/or the aptamer 350 may be coupled to a solid support (e.g., bead, substrate) such that the second double-stranded region 388 is cleaved off from the solid support. In an embodiment, the arrangement may be reversed such that the second double-stranded region 388 is coupled to the solid support, and cleaves releases the aptamer 350 and aptamer binding region 360, which can be removed by washing.

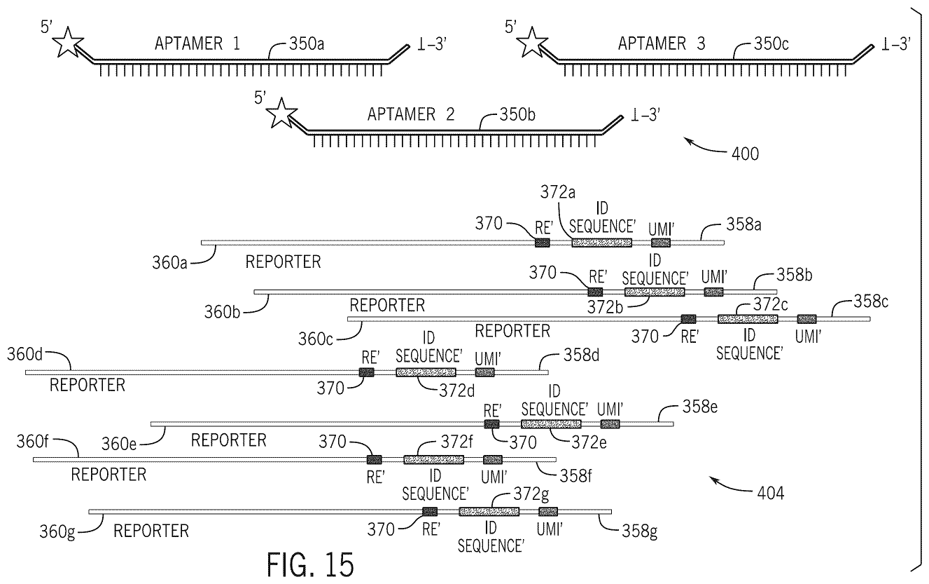

In FIG. 14, a single aptamer type of an individual aptamer 350 is shown. It should be understood that the illustrated workflow may be extended to all aptamers in a multiplexed aptamer-based assay in parallel. Further, the assay eluate may include multiple aptamers 350, which is dependent on the concentration of the target molecule of the aptamer 350 in the assessed sample. The aptamer 350 is a single-stranded nucleic acid having a fixed or substantially fixed nucleic acid sequence. Thus, copies or multiples of the individual aptamer 350 may all share a conserved sequence. In an embodiment, aptamers 350 may include modified nucleotides. Different aptamers, referred to generally as aptamers 350, may have different nucleic acid sequences relative to one another, which facilitates different target specificity for respective different aptamers 350, as generally illustrated in FIGS. 15-20.

FIG. 15 shows a workflow that includes the step of contacting a group 400 of different aptamers 350a, 14b, 14c with a panel 404 of reporter probes 358a, 358b, 358c, 358d, 358e, 358f, 24g. The group 400 may be generated from an aptamer-based assay that uses a fixed or known population of aptamers 350. After the aptamer-based assay, only a subset of the aptamers 350 are retained based on their binding to analytes present in the sample, which is represented by the group 100. For an uncharacterized sample, the concentration and/or type of aptamers 350 present in the sample eluate or solution may be unknown before detection.

The illustrated group 400 and panel 404 are by way of example, and it should be understood that more or fewer aptamers 350 and/or reporter probes 358 may be present. In an example, the panel 404 may be a commercial panel with a fixed number or composition of different types of reporter probes 358 specific for aptamers 350 of interest. In an embodiment, the panel 404 represents reporter probes 358 specific for all aptamers 350 used in the assay. In an embodiment, the aptamer-based assay uses at least 100, 500, 1000, or 50000 different aptamers, and the panel 404 has a corresponding number of different reporter probes 358.

The length of the reporter probes 358 may be based on the length of the individual aptamers 350. In an embodiment, the reporter probes 358 have a same length or have variable lengths with respect to one another within the panel 404. In an embodiment, the reporter probes are 30-200 nucleotides in length. The panel 404 may be provided as a mixture of the different reporter probes in a single-stranded state. Thus, the sequences of the reporter probes 358 may be designed to avoid cross-complementarity.

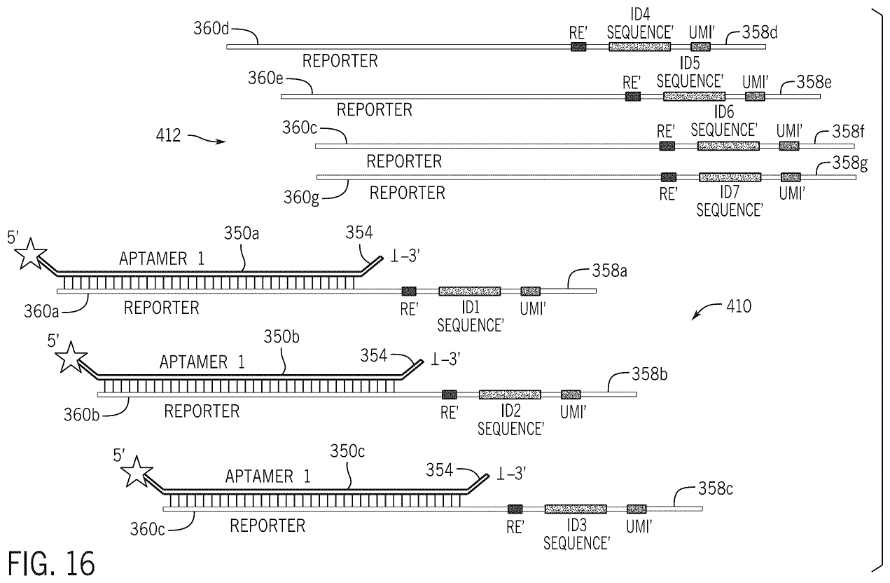

FIG. 16 shows hybridization of the reporter probes 358a, 358b, 358c specific for the aptamers 350a, 350b, 350c of the group 100 to create aptamer-reporter probe complexes 410. Other reporter probes 358d, 358e, 358f, 358g do not have corresponding aptamers 350 in the group 100 and, therefore, create a single-stranded reporter probe subset 412.

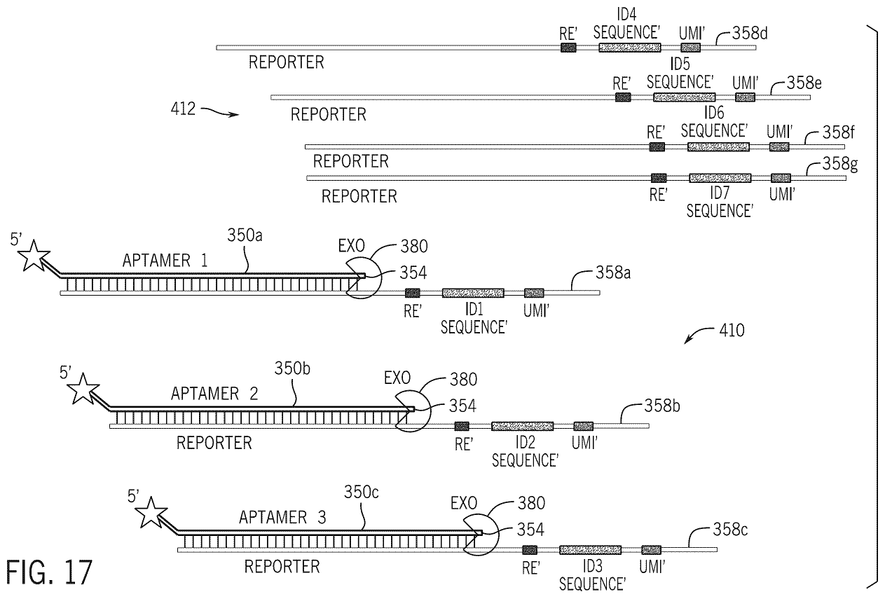

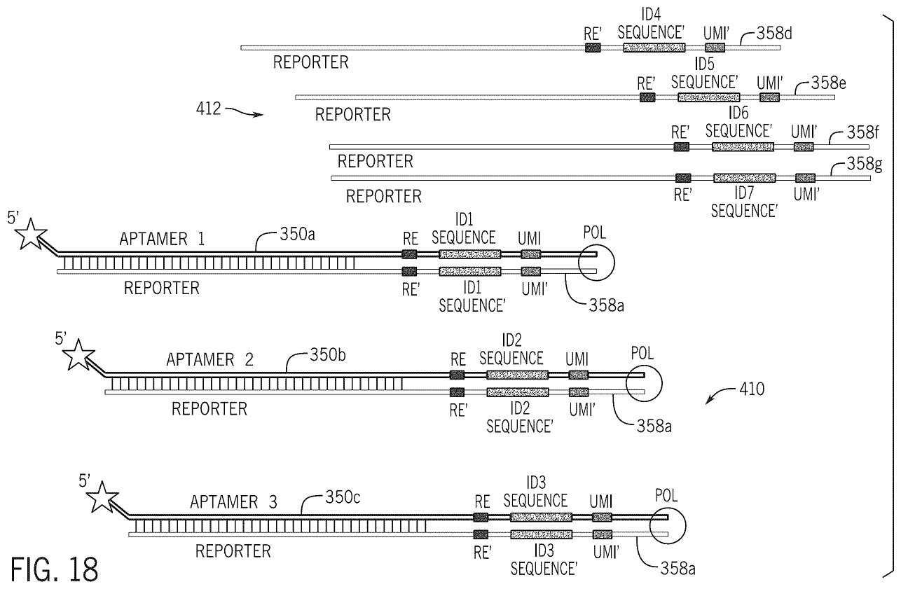

FIG. 17 shows deprotection of 3′ end 354 for the reporter probes 358a, 358b, 358c in complexes 410 using an exonuclease 380, by way of example. As discussed herein, the deprotection may include a heat inactivation step. However, in other embodiments, the reaction may be isothermal. Further, in certain embodiments, the deprotection may be accomplished using a DNA polymerase with exonuclease activity (such as DNA Pol 1). FIG. 18 shows the aptamer-reporter probe complexes 410 after extension from the deprotected ends 354 (FIG. 17) to create double-stranded structures. Accordingly, the aptamers 350a, 14b, 14c are extended using the sequence of the reporter probes 358a, 358b, 358c as a template. This creates a strand having sequences complementary to the full-length of the reporter probes 358a, 358b, 358c.

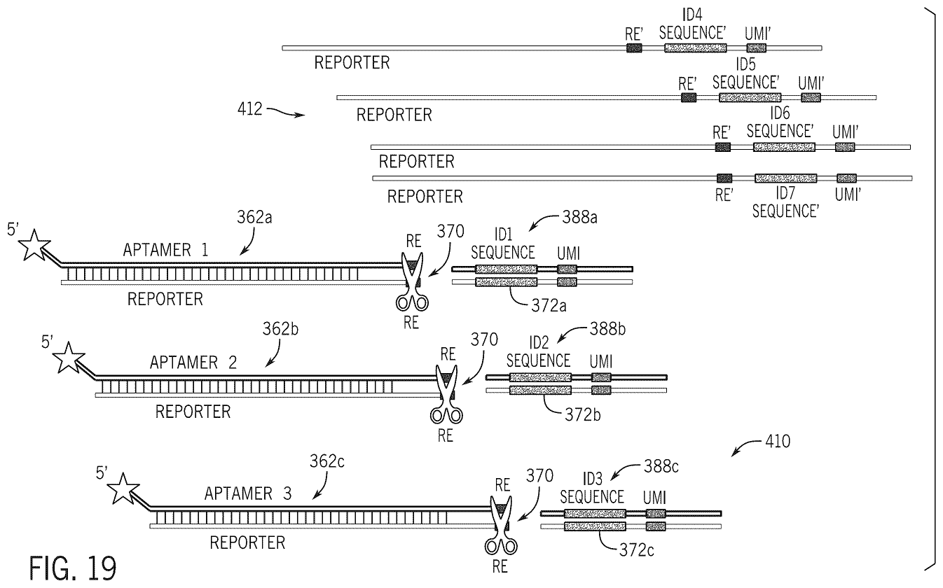

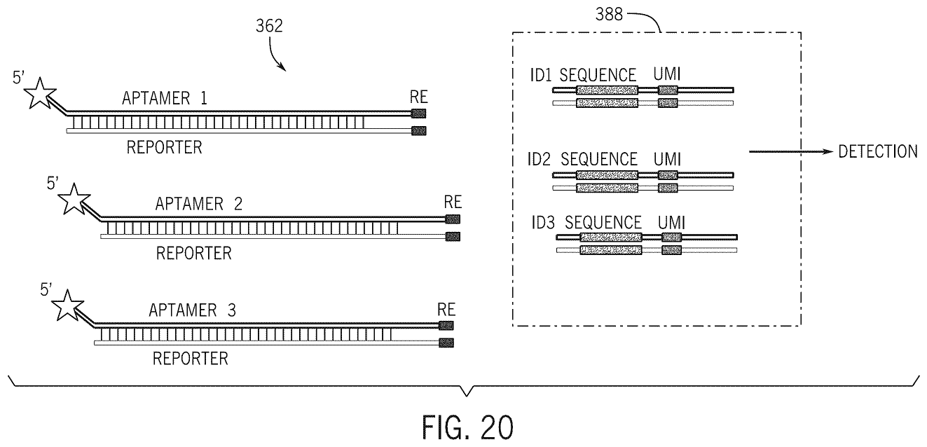

As shown in FIG. 19, cleavage at the cleavage region 370 separates a first double-stranded region 36a, 36b, 36c from a second double-stranded region 388a, 388b, 388c of each complex 410. Remaining single-stranded reporter probes 358 of the unbound subset 412 can be digested such that only double-stranded structures remain, as shown in FIG. 20. After digestion of any unbound reporter probes 358, the second-double stranded regions 388 can be processed and detected. Digestion or removal of the unbound reporter probes 358 of the subset 412 prevents false positives at later detection steps.

As noted, the second-double stranded regions 388 can be separated by elution or by using a solid support. However, in certain embodiments, no separation step may be performed, because only the second-double stranded regions 388 may be operable substrates for downstream library preparation steps. For example, the cleaved end may be a substrate for a sequencing adapter. In an embodiment, the second-double stranded regions 388 may include primer binding regions that permit incorporation of adapters via amplification. In an embodiment, the second-double stranded regions 388 may be captured using an affinity tag, such as a capture bead coupled to an affinity tag binder. In one embodiment, each reporter probe 358 may be provided with an affinity tag. However, other arrangements are also contemplated, including column-based, flow-cell based, or substrate-based separation using a capture entity that binds to the affinity tag. After the separation, the sample proceeds to sequence library preparation steps, shown as a ligation to PCR reaction. However, other preparation workflows are also contemplated, such as direct amplification, step out PCR, or other amplification and/or ligation preparations as discussed herein. The end products of the workflow include oligonucleotide fragments that can then be sequenced as part of a sequencing reaction to generate sequence data.

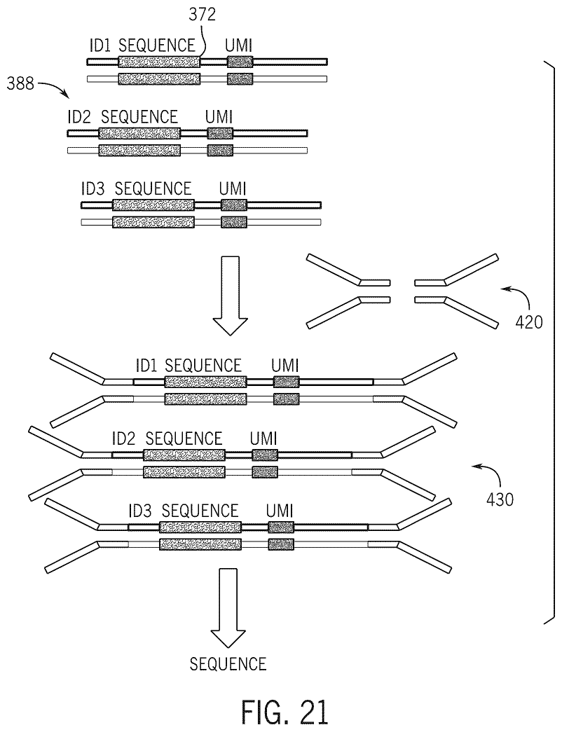

FIG. 21 shows an example adapterization technique that can be used to conform the separated double-stranded region 388 including the identification sequence 372 into inputs for sequencing library preparation or, in embodiments, into a sequencing library including adapterized fragments 220 that can be sequenced to generate sequence data. Accordingly, the disclosed embodiments may, in embodiments, provide an advantage of incorporating one or more sequencing library preparation steps into the detection of the aptamer 350. Further, the disclosed embodiments may permit certain steps of sequencing library preparation to be omitted or combined, thus increasing detection efficiency. In embodiments, the disclosed embodiments are also directed to sequencing techniques that permit generation of sequence data from sequence reads.

The adapters 420 may be incorporated onto the separated double-stranded region 388 via ligation and/or via one or more amplification steps. In certain embodiments, the reporter probe 358 may include conserved primer regions that are used for later adapter incorporation via amplification.

Embodiments of the present disclosure include aptamer indirect detection via a deprotect and extend technique, as generally shown and discussed with respect to FIG. 2. During extension, a biontylated or affinity tagged nucleotide or sequence is added to 3′ end of the aptamer, which permits a hybridization capture via the tag. Detection may occur using reporter probes as discussed.

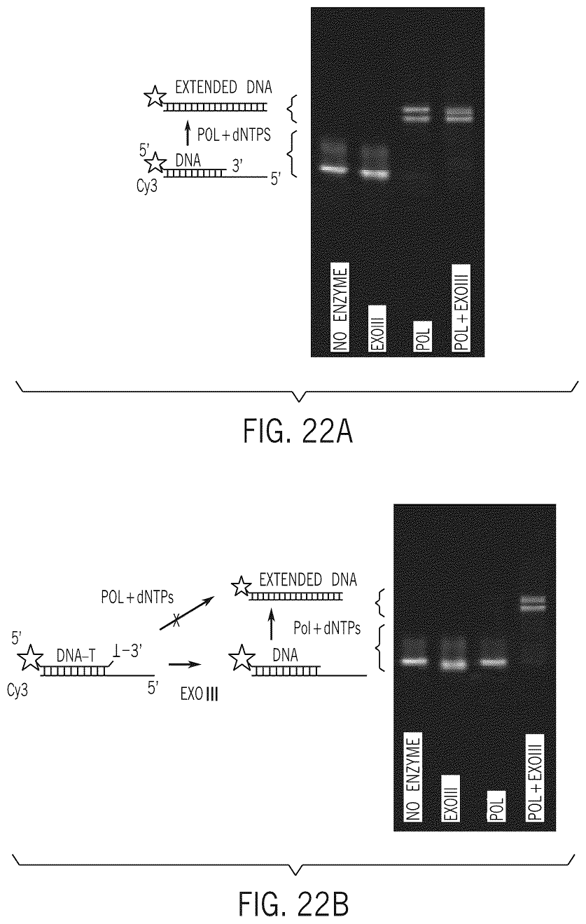

FIG. 22A and FIG. 22B show results from an assay demonstrating that the inverted dT acts to protect 3′ end of the aptamer from polymerase extension when in place. Table 1 shows assay conditions. In certain cases, the aptamers may be in a sodium perchlorate buffer that inhibits enzyme activity. In this case, the reaction volume should be increased to dilute the perchlorate buff, and the reaction buffer components may be adapted to have low salt (e.g., NaCl) or ionic strength.

TABLE 1
Assay Conditions for Aptamer extension

Hybridization mix component

| | |
|---|---|
| DNA/aptamers/SOMAmers with 3′ | May be in a buffer containing |
| inverted dT Buffer | sodium perchlorate |
| | Hybridisation buffer may |
| | contain additives to promote |
| | hybridization |
| DNA reporters to hybridise to | Reporters should in excess, |
| SOMAmers, with a unique | e.g. in 10-fold excess over the |
| PCR-amplifiable barcode ID | SOMAmer concentration to |
| | promote hybridisation. |
| | Reporters may be designed |
| | with the barcode ID at the 3′ or |
| | 5′ end of the reporter. |
| Denaturisation step | For example, 95° C. for 10 |
| Ramp to hybridisation temperature | minutes |
| Hybridization | Ramp at −0.1° C./s |
| | E.g. 48° C. for 19 hr |
| | (These conditions should be |
| | adapted for the input DNA |
| | being used in the assay. |
| | Conditions should be chosen |
| | to achieve highest percentage |
| | of SOMAmers hybridized to |
| | reporters) |

ExoIII can remove inverted dT from 3′ ends of DNA oligonucleotides. 5′-Cy3 DNA oligos were hybridized to longer unlabeled oligos such that the Cy-3-labeled oligo was double-stranded and the unlabeled oligo had a 5′-overhang. After enzymatic treatment, products were analyzed by 10% TBE-UREA gel electrophoresis with Cy3 detection. In FIG. 22A, a hybridized 5′-Cy3 DNA oligo with no 3′ modification (DNA) can be extended by polymerase (Pol) with dNTPS. Treatment with 3′ to 5′ exonuclease ExoIII under these reaction conditions is insufficient to completely digest DNA oligos or extended DNA. In FIG. 22B, a hybridized 5′-Cy3 DNA oligo with a 3′ inverted dT (DNA-T) is unable to be extended by polymerase (Pol) with dNTPS. Treatment with 3′ to 5′ exonuclease ExoIII and polymerase allows DNA extension via removal of 3′ inverted dT by ExoIII to allow polymerase extension. 5′-Cy3 oligos run on gel as a band of ssDNA Cy-3 labeled oligo, and a higher band of dsDNA Cy-3 labeled oligo: extension oligo.

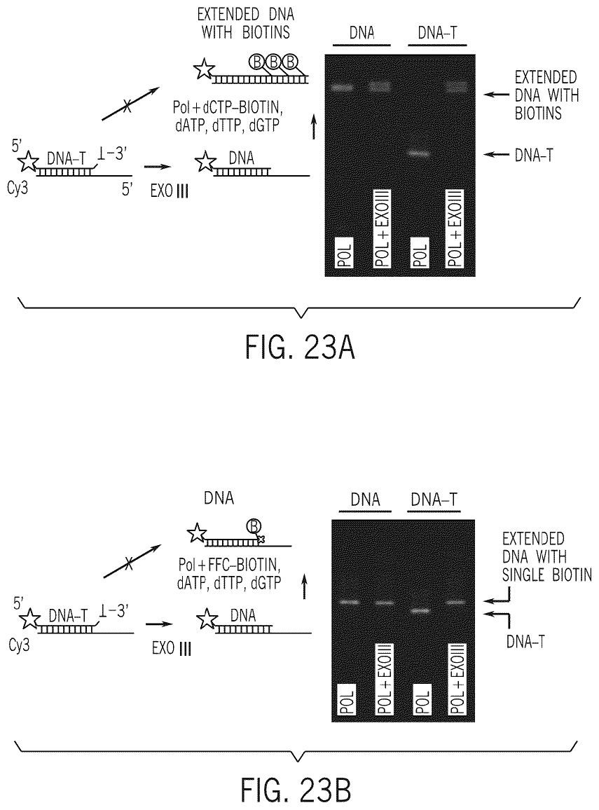

FIG. 23A shows data from a model system demonstrating that DNA with an inverted dT can be deprotected by ExoIII and extended by a polymerase with multiple biotinylated nucleotides and is inert to extension without deprotection. FIG. 23B shows data from a model system demonstrating that DNA with an inverted dT can be deprotected by ExoIII and extended by a polymerase with a biotinylated nucleotide and is inert to extension without deprotection. 5Cy-3 DNA oligos were hybridized to linger unlabeled oligos such that the Cy3-labeled oligo is double-stranded and the unlabeled oligo has a 5′ overhang. After enzymatic treatment, products were analyzed by 10% TBE-UREA gel electrophoresis with Cy3 detection. The polymerase Pol can incorporated biotinylated dNTPs such as dCTP-biotin (FIG. 23A) or biotinylated dNTPs with a 3′ block, such as FFC-biotin (FIG. 23B), resulting in extended DNA with multiple biotinylated nucleotides or a single terminal biotinylated nucleotide, respectively. The polymerase is only able to extend a DNA oligo with a 3′ inverted dT after ExoIII deprotection.

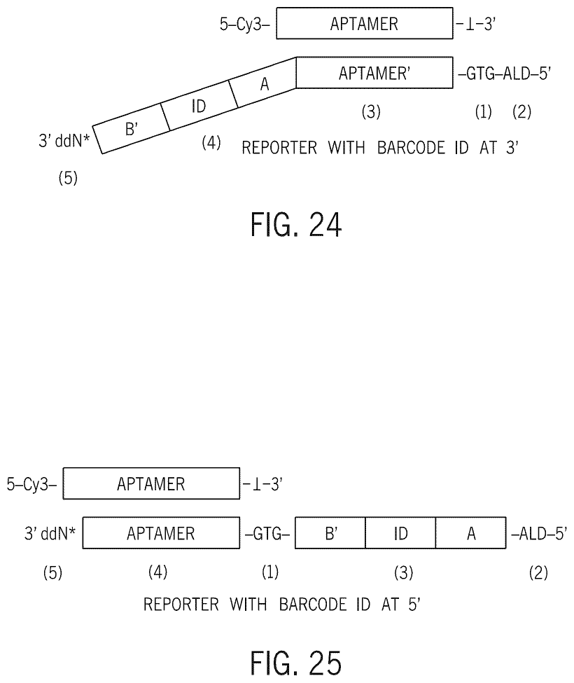

FIG. 24 shows an example reporter with an identification sequence or ID at a 3′ end, and FIG. 25 shows an example reporter with an identification sequence or ID at a 5′ end. Reporter probes may hybridize to aptamers with an overhang at the 5′ end of the reporter, shown as (1) in the illustrated figures. The 5′ overhang may include a base complementary to the biotinylated nucleotide incorporated. For example, the sequence may include a G to incorporate a biotinylated FFC-biotin in the enzyme reaction mix. Reporters may be purified after synthesis to ensure full-length. A 5′ aldehyde (shown as reference (2)) may be incorporated at the reporter 5′ end to facilitate reporter purification. Reporter hybridization regions may be a full length of the aptamer or shorter to facilitate manufacturing accuracy. In an embodiment, the identification sequences may be flanked by adapter sequences to facilitate PR-based library preparation techniques for NGS (shown as references (3 (and (4)). Reporters may be protected at the 3′ end to prevent modification during the assay, e.g., direct biotinylation of the reporter by the polymerase if 3′ end becomes double-stranded, or digestion by the exonuclease. The protection maybe a dideoxynucleotide added to the 3′ end (shown as reference (5)) via a RP-isomer phosphorothioate linkage (ddN*), which would resist both ExoIII digestion and DNA polymerase extension.

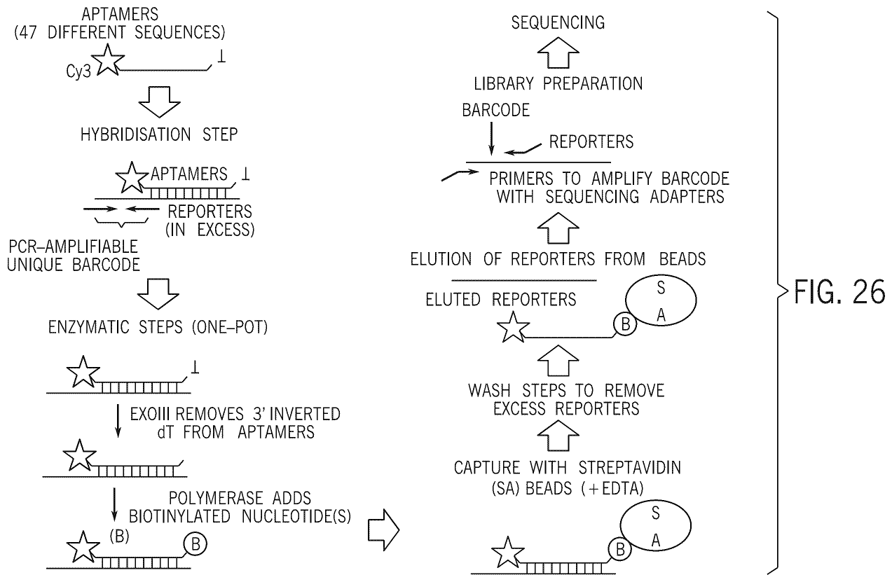

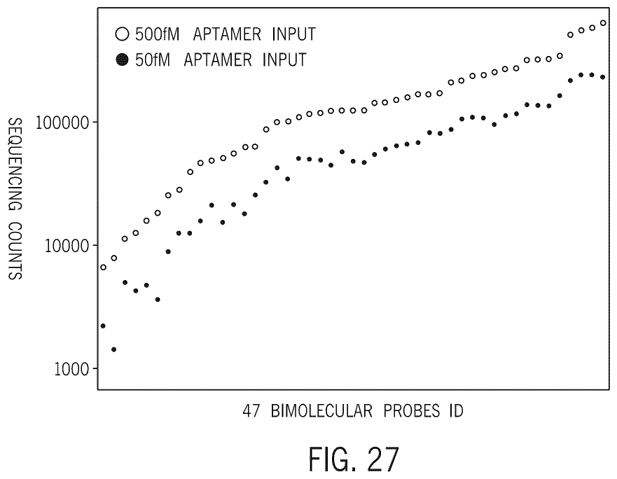

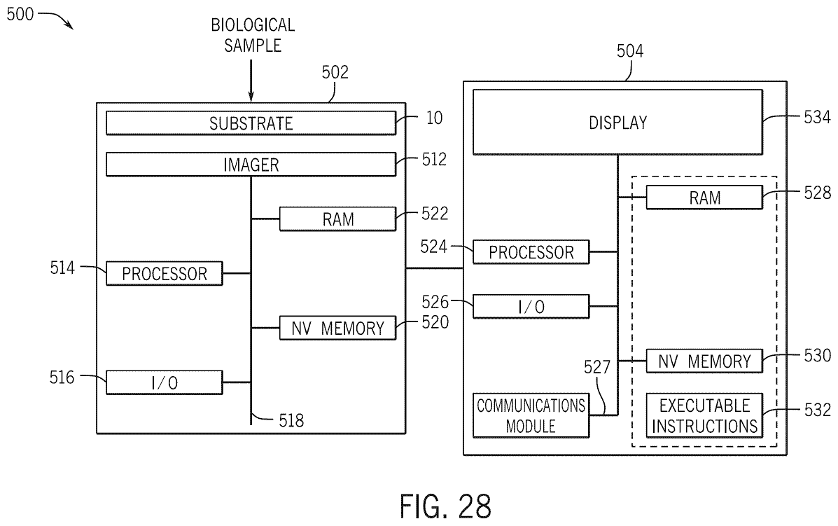

FIG. 26 shows a deprotect and extend workflow. FIG. 27 shows proof-of-concept with aptamers showing that 3′ inverted dT can be removed and that, after removal, extension with biotinylated nucleotides occurs. Deprotect and extend reaction considerations are shown in Table 2.

TABLE 2
Deprotect and extend reaction considerations

| | |
|---|---|
| Enzymatic | |
| reaction mix | |
| component | |
| ExoIII | ExoIII at a concentration low enough to prevent |
| | excessive (or complete) degradation of DNA under the |
| | reaction conditions, but sufficient to excise at least |
| | one base from every SOMAmer. An example |
| | concentration is 0.04 U/ul reaction of ExoIII. |
| DNA | Such as Pol2511 (Illumina), at a high enough |
| polymerase | concentration to fully incorporate all DNA strands e.g. |
| | approx. 2.7 uM |
| Biotinylated | Such as 2 uM FFC-biotin (FFC-(N3)2-LC-BIOTIN, |
| nucleotide with | Illumina), either alone or with other biotinylated |
| 3′OH block | nucleotides with 3′OH blocks. |
| Optional - other | Master mix may include 2 uM each dTTP, dATP and |
| nucleotides | dGTP, or no additional dNTPs. |
| Mg²⁺ | Such as 4 mM MgSO₄ |
| Buffer | Such as 10 mM ethanolamine buffer pH 9.9 at 25 C. |
| | Should not include NaCl or high salt/ionic strength |
| | components if sodium perchlorate is present in the |
| | reaction. |
| Water | Sufficient to dilute inhibitors that may be present in the |
| | hybridisation mix. For example, to prevent sodium |
| | perchlorate buffer (in the buffer from SOMAscan |
| | assay) inhibiting ExoIII or polymerase activity, it can |
| | be diluted e.g. 10-fold. |
| Enzymatic | |
| reaction | |
| conditions | |
| Deprotect step | For example, 37° C. for 10 min |
| Extension step | 37-68° C. for X min - (for example 50° C. for 30 min |
| | with Pol2511) |

In the illustrated workflows, a single aptamer type of an individual aptamer, e.g., the aptamer 20 and/or the aptamer 350, is shown. Further, the assay eluate may include multiple aptamers. The aptamer may be a single-stranded nucleic acid having a fixed or substantially fixed nucleic acid sequence. Thus, copies or multiples of the individual aptamer may all share a conserved sequence. Different aptamers, referred to generally as aptamers, may have different nucleic acid sequences relative to one another, which facilitates different target specificity for respective different aptamers.

In certain embodiments of the disclosure, the disclosed probes can include one or more conserved regions, such as a conserved primer region, e.g., a first conserved primer region and a second conserved primer region. A conserved region is conserved between at least some other probes of a probe set such that the conserved region has an identical or similar nucleotide sequence as compared between the probes.

One or more probes as discussed herein may include an affinity tag. Affinity tags can be useful for a variety of applications, for example the bulk separation of target nucleic acids hybridized to hybridization tags. As used herein, the term “affinity tag” and grammatical equivalents can refer to a component of a multi-component complex, wherein the components of the multi-component complex specifically interact with or bind to each other. For example an affinity tag can include biotin or poly-His that can bind streptavidin or nickel, respectively. Other examples of multiple-component affinity tag complexes are listed, for example, U.S. Patent Application Pub. No. 2012/0208705, U.S. Patent Application Pub. No. 2012/0208724 and Int. Patent Application Pub. No. WO 2012/061832, each of which is incorporated by reference in its entirety.

The disclosed embodiments may include compositions of one or more primers and/or probes. Probes and/or primers of the disclosed embodiments are designed to be complementary to a target sequence (either the target sequence of the sample or to other probe sequences), such that hybridization of the target sequence and the probes of the present invention occurs. As outlined below, this complementarity need not be perfect; there may be any number of base pair mismatches which will interfere with hybridization between the target sequence and the single stranded nucleic acids of the present invention. However, if the number of mutations is so great that no hybridization can occur under even the least stringent of hybridization conditions, the sequence is not a complementary target sequence. Thus, by “substantially complementary” herein is meant that the probes are sufficiently complementary to the target sequences to hybridize under normal reaction conditions.

A variety of hybridization conditions may be used in the present invention, including high, moderate and low stringency conditions. Longer sequences hybridize specifically at higher temperatures. Generally, stringent conditions are selected to be about 5-10° C. lower than the thermal melting point (Tm) for the specific sequence at a defined ionic strength and pH. The Tm is the temperature (under defined ionic strength, pH and nucleic acid concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g. 10 to 50 nucleotides) and at least about 60° C. for long probes (e.g. greater than 50 nucleotides).

In certain embodiments, probe contacting steps may be run under stringency conditions which allows formation of the hybridization complex only in the presence of target. Stringency can be controlled by altering a step parameter that is a thermodynamic variable, including, but not limited to, temperature, formamide concentration, salt concentration, chaotropic salt concentration, pH, organic solvent concentration, etc. The size of the primer nucleic acid may vary, as will be appreciated by those in the art, in general varying from 5 to 500 nucleotides in length. Primers may be between 10 and 100, between 15 and 50, and from 10 to 35 depending on the use and amplification technique.

In certain embodiments, the reporter probes 358 are single-stranded or are part of a double-stranded structure that includes a restriction enzyme cleavage site with a sequence cleavable by a restriction enzyme. The restriction enzyme can be a Type I enzyme (EC 3.1.21.3), a Type II enzyme (EC 3.1.21.4), a Type III enzyme (EC 3.1.21.5), or a Type IV enzyme (EC 3.1.21.5). Restriction enzymes can include, by way of example, Alu I, Ava I, Bam HI, Bgl II, Eco P15 I, Eco RI, Eco RII, Eco RV, Hae III, Hga I, Hha I, Hind III, Hinf I, Hpa I, Kpn I, Mbo I, Not I, Pst I, Pvu II, Sac I, Sal I, Sau 3A, Sca I, Sma I, Spe I, Sph I, Sst I, Stu I, Taq I, Xba I or Xma I. The restriction enzyme can be a recombinant restriction enzyme. In an embodiment, the restriction enzyme cleavage is a blunt-ended cleavage. In an embodiment, the restriction enzyme cleavage is a sticky-ended cleavage. Thus, the cleavage region 40 may, when double-stranded, form a double-stranded restriction site.

In an embodiment, a cleavage region, such as a cleavage region 370, may, when double-stranded, form a cleavage site for a CRISPR-Cas system using a guide RNA. CRISPR-Cas systems provided herein include engineered and/or programmed nuclease systems derived from naturally occurring CRISPR-Cas systems. In an embodiment, the CRISPR-Cas system is a CRISPR-Cas9 system. CRISPR-Cas systems may include contain engineered and/or mutated Cas proteins. CRISPR-Cas systems may also contain engineered and/or programmed guide RNA. In some embodiments, the crRNA or the derivative thereof provided herein is a polynucleotide having a crRNA polynucleotide fused to a tracrRNA polynucleotide. In one embodiment, the Cas protein or the variant thereof provided herein can be directed by a chimeric sgRNA to any genomic locus followed by a 5′-NGG protospacer-adjacent motif (PAM). For example, in some embodiments, crRNA and tracrRNA are synthesized by in vitro transcription, using a synthetic double stranded DNA template containing the T7 promoter. The tracrRNA has a fixed sequence, whereas the target sequence dictates part of crRNA's sequence. Equal molarities of crRNA and tracrRNA are mixed and heated at 55° C. for 30 seconds. Cas9 is added at the same molarity at 37° C. and incubated for 10 minutes with the RNA mix. 10-20 fold molar excess of Cas9 complex is then added to the target DNA. The binding reaction can occur within 15 minutes.

The disclosed techniques are directed to aptamer detection in one or more applications, such as for analysis of an eluate of an aptamer-based assay. The techniques may be part of sequencing library preparation that may include oligonucleotide adapters, e.g., that may be coupled to reporter probes, for downstream sequencing. The adapters may be integral to the probes and/or may be coupled in any other suitable manner. In some embodiments, the adapters are introduced in a multi-step process, such as a two-step process, involving ligation of a portion of the adapter to the target polynucleotide having a universal primer sequence. The second step includes extension, for example by PCR amplification, using primers that include a 3′ end having a sequence complementary to the attached universal primer sequence and a 5′ end that contains other sequences of an adapter. By way of example, such extension may be performed as described in U.S. Pat. No. 8,053,192, which is hereby incorporated by reference in its entirety. Additional extensions may be performed to provide additional sequences to 5′ end of the resulting previously extended polynucleotide.

In some embodiments, the adapter may be ligated to the reporter probes. Any suitable adapter may be attached to a target polynucleotide, such as a reporter probe, via any suitable process, such as those discussed herein. The adapter can include a library-specific index tag sequence (e.g., i5, i7). The index tag sequence may be attached to the target polynucleotides from each library before the sample is immobilized for sequencing. The index tag is not itself formed by part of the target polynucleotide, but becomes part of the template for amplification. The index tag may be a synthetic sequence of nucleotides which is added to the target as part of the template preparation step. Accordingly, a library-specific index tag is a nucleic acid sequence tag which is attached to each of the target molecules of a particular library, the presence of which is indicative of or is used to identify the library from which the target molecules were isolated. In an embodiment, the index tag sequence is 20 nucleotides or less in length. For example, the index tag sequence may be 1-10 nucleotides or 4-6 nucleotides in length. A four nucleotide index tag gives a possibility of multiplexing 256 samples on the same array, a six base index tag enables 4,096 samples to be processed on the same array. The adapters may contain more than one index tag so that the multiplexing possibilities may be increased.

The adapters may include any other suitable sequence in addition to the index tag sequence. For example, the adapters may include universal extension primer sequences, which are typically located at 5′ or 3′ end of the adapter and the resulting polynucleotide for sequencing. The universal extension primer sequences may hybridize to complementary primers bound to a surface of a solid substrate. The complementary primers include a free 3′ end from which a polymerase or other suitable enzyme may add nucleotides to extend the sequence using the hybridized library polynucleotide as a template, resulting in a reverse strand of the library polynucleotide being coupled to the solid surface. Such extension may be part of a sequencing run or cluster amplification.

In some embodiments, the adapters include one or more universal sequencing primer sequences. The universal sequencing primer sequences may bind to sequencing primers to allow sequencing of an index tag sequence, a target sequence, or an index tag sequence and a target sequence. In some embodiments, the disclosed reporter probes, e.g., reporter probe 24, may include a “sequencing adaptor” or “sequencing adaptor site”, that is to say a region that comprises one or more sites that can hybridize to a primer. In some embodiments, a sequence can include at least a first primer site useful for amplification, sequencing, and the like.

After adapter incorporation, the disclosed reporter probes may be sequenced. In one example, the sequencing may be via Illumina's sequencing-by-synthesis and reversible terminator-based sequencing chemistry. Illumina's sequencing technology relies on the attachment of fragmented genomic DNA to a planar, optically transparent surface on which oligonucleotide anchors are bound. Template DNA is end-repaired to generate 5′-phosphorylated blunt ends, and the polymerase activity of Klenow fragment is used to add a single A base to the 3′ end of the blunt phosphorylated DNA fragments. This addition prepares the DNA fragments for ligation to oligonucleotide adapters, which have an overhang of a single T base at their 3′ end to increase ligation efficiency. The adapter oligonucleotides are complementary to the flow-cell anchors. Under limiting-dilution conditions, adapter-modified, single-stranded template DNA is added to the flow cell and immobilized by hybridization to the anchors. Attached DNA fragments are extended and bridge amplified to create an ultra-high density sequencing flow cell with hundreds of millions of clusters, each containing ˜1,000 copies of the same template. In one embodiment, the randomly fragmented genomic DNA is amplified using PCR before it is subjected to cluster amplification. Alternatively, an amplification-free genomic library preparation is used, and the randomly fragmented genomic DNA is enriched using the cluster amplification alone. The templates are sequenced using a robust four-color DNA sequencing-by-synthesis technology that employs reversible terminators with removable fluorescent dyes. High-sensitivity fluorescence detection is achieved using laser excitation and total internal reflection optics. Sequence are aligned against a truth table or stored correlations between aptamer identity and identification sequences using specially developed data analysis pipeline software.

In some embodiments, one or more nucleic acids, such as an aptamer 14 or reporter probe 24, may include a label or dye that operates as a detection moeity. In some embodiments, a detection moiety is a hapten that is detectable via a binding partner-fluorescent moiety conjugate. In some embodiments, a rbNTP conjugate comprises one or both of a fluorescent moiety and a hapten linked to a rbNTP via one or more linkers. In some embodiments a hapten is a biotin, digoxigenin (DIG) or dinitrophenol (DNP). In some embodiments, a hapten is detected by a binding partner-fluorescent moiety conjugate. In some embodiments, a binding partner is a small molecule or an antibody or fragment thereof, for example streptavidin, anti-DIG or anti DNP. In some embodiments, a detection moiety is an optically detectable moiety, such as a fluorescent moiety, or derivatives thereof. Optically-detectable moieties in accordance with disclosed embodiments include, but are not limited to, fluorescein and fluorescein derivatives such as carboxyfluorescein, tetrachlorofluorescein, hexachlorofluorescein, carboxynapthofluorescein, fluorescein isothiocyanate, NHS-fluorescein, iodoacetamidofluorescein, fluorescein maleimide, SAMSA-fluorescein, fluorescein thiosemicarbazide, carbohydrazinomethylthioacetyl-amino fluorescein, rhodamine and rhodamine derivatives such as TRITC, TMR, lissamine rhodamine, Texas Red, rhodamine B, rhodamine 6G, rhodamine 10, NHS-rhodamine, TMR-iodoacetamide, lissamine rhodamine B sulfonyl chloride, lissamine rhodamine B sulfonyl hydrazine, Texas Red sulfonyl chloride, Texas Red hydrazide, coumarin and coumarin derivatives such as AMCA, AMCA-NHS, AMCA-sulfo-NHS, AMCA-HPDP, DCIA, AMCE-hydrazide, BODIPY and derivatives such as BODIPY FL C3-SE, BODIPY 530/550 C3, BODIPY 530/550 C3-SE, BODIPY 530/550 C3 hydrazide, BODIPY 493/503 C3 hydrazide, BODIPY FL C3 hydrazide, BODIPY FL IA, BODIPY 530/551 IA, Br-BODIPY 493/503, Cascade Blue and derivatives such as Cascade Blue acetyl azide, Cascade Blue cadaverine, Cascade Blue ethylenediamine, Cascade Blue hydrazide, Lucifer Yellow and derivatives such as Lucifer Yellow iodoacetamide, Lucifer Yellow CH, cyanine and derivatives such as indolium based cyanine dyes, benzo-indolium based cyanine dyes, pyridium based cyanine dyes, thiozolium based cyanine dyes, quinolinium based cyanine dyes, imidazolium based cyanine dyes, Cy 3, Cy5, lanthanide chelates and derivatives such as BCPDA, TBP, TMT, BHHCT, BCOT, Europium chelates, Terbium chelates, Alexa Fluor dyes, DyLight dyes, Atto dyes, LightCycler Red dyes, CAL Flour dyes, JOE and derivatives thereof, Oregon Green dyes, WellRED dyes, IRD dyes, phycoerythrin and phycobilin dyes, Malacite green, stilbene, DEG dyes, NR dyes, and/or near-infrared dyes.

The adapters disclosed herein may include universal or conserved sequences that can be used in conjunction with Illumina® sequencing reactions. It should be understood that these are by way of example, and any of the disclosed arrangements may be used in conjunction with disclosed techniques. The adapters may include adapter sequence, such as examples of sequences, or their complements, for primer 1 and primer 2 used in Illumina® sequencing preparations, A14, B15, during amplification. In other embodiments, universal capture primer sequences and/or sample index sequences can be incorporated into oligonucleotides generated from the reporter probes 358, such as via amplification and/or ligation and extension. Certain arrangements that include indexes may incorporate a custom or bridged primer during sequencing to accommodate different indexes. Other embodiments may include custom options for sequencing libraries using single reads from surface P5 for example, or for adding dark sequencing by synthesis cycles where common sequences exist in adapter regions.

The adapter sequences A14-ME, ME, B15-ME, ME′, A14, B15, and ME are provided below and may be used alone or in combination with the disclosed adapters. In certain embodiments, the adapter sequences may be incorporated directly into the reporter probes 358. Example adapter sequences are provided and may be used in conjunction with the disclosed embodiments as part of library preparation techniques. However, other adapter sequences are also contemplated. The adapter sequences A14-ME, ME, B15-ME, ME′, A14, B15, and ME are provided below:

A14-ME:
(SEQ ID NO: 1)
5′-TCGTCGGCAGCGTCAGATGTGTATAAGAGACAG-3′
B15-ME:
(SEQ ID NO: 2)
5′-GTCTCGTGGGCTCGGAGATGTGTATAAGAGACAG-3′
ME′:
(SEQ ID NO: 3)
5′-phos-CTGTCTCTTATACACATCT-3′
A14:
(SEQ ID NO: 4)
5′-TCGTCGGCAGCGTC-3′
B15:
(SEQ ID NO: 5)
5′-GTCTCGTGGGCTCGG-3′
ME:
(SEQ ID NO.: 6)
AGATGTGTATAAGAGACAG

The modified aptamers and/or reporter probes as disclosed herein can include a region having the sequence of a universal Illumina® capture primer or a region specifically hybridizing with a universal Illumina® capture primer. Universal Illumina® capture primers include, e.g., P5 5′-AATGATACGGCGACCACCGA-3′ ((SEQ ID NO: 7)) or P7 (5′-CAAGCAGAAGACGGCATACGA-3′ (SEQ ID NO: 8)), or fragments thereof. A region specifically hybridizing with a universal Illumina® capture primer can include, e.g., the reverse complement sequence of the Illumina® capture primer P5 (“anti-P5”: 5′-TCGGTGGTCGCCGTATCATT-3′ (SEQ ID NO: 9) or P7 (“anti-P7”: 5′-TCGTATGCCGTCTTCTGCTTG-3′ (SEQ ID NO:10)), or fragments thereof.

A conserved primer region can additionally or alternatively include a region having the sequence of an Illumina® sequencing primer, or fragment thereof, or a region specifically hybridizing with an Illumina® sequencing primer, or fragment thereof. Illumina® sequencing primers include, e.g., SBS3 (5′-ACACTCTTTCCCTACACGACGCTCTTCCGATCT-3′ (SEQ ID NO: 11)) or SBS8 (5′-CGGTCTCGGCATTCCTGCTGAACCGCTCTTCCGATCT-3′ (SEQ ID NO: 12)). A region specifically hybridizing with an Illumina® sequencing primer, or fragment thereof, can include, e.g., the reverse complement sequence of the Illumina® sequencing primer SBS3 (“anti-SBS3”: 5′-AGATCGGAAGAGCGTCGTGTAGGGAAAGAGTGT-3′ (SEQ ID NO: 13)) or SBS8 (“anti-SBS8”: 5′-AGATCGGAAGAGCGGTTCAGCAGGAATGCCGAGACCG-3′ (SEQ ID NO: 14)), or fragments thereof. The incorporation of sequencing primer sequences in the reporter probes may be either directly or via subsequent amplification, ligation, or other sequencing library preparation steps.

In an embodiment, the disclosed amplification products may include amplification products that differ from one another based on different identification sequences but that have conserved or universal primer regions. In this manner, a single primer set can be used to amplify oligonucleotides that have variable identification sequences. Provided herein are library preparation kits that include primers that are capable of generating amplification products with incorporated adapters to generate sequencing libraries. The kits may include aptamer-binding oligonucleotides and corresponding primers as discussed herein.

In an embodiment, the sequencing may use Illumina® NGS primers. The following primers are shown by way of example.

Read 1
(SEQ ID NO: 15)
5′ TCGTCGGCAGCGTCAGATGTGTATAAGAGACAG 3′
Read 2
(SEQ ID NO: 16)
5′ GTCTCGTGGGCTCGGAGATGTGTATAAGAGACAG
Paired End Read 1
(SEQ ID NO: 17)
5′ ACACTCTTTCCCTACACGACGCTCTTCCGATCT
Paired End Read 2
(SEQ ID NO: 18)
5′ CGGTCTCGGCATTCCTGCTGAACCGCTCTTCCGATCT
Index 1 Read
(SEQ ID NO: 19)
5′ CAAGCAGAAGACGGCATACGAGAT[i7]GTCTCGTGGGCTCGG
Index 2 Read
(SEQ ID NO: 20)
5′ AATGATACGGCGACCACCGAGATCTACAC[i5]TCGTCGGCAGCGTC

It should be understood that the index read primers may be designed to include the particular index sequence associated with a particular sample in an aptamer-based assay. Thus, the index primers may have a nucleotide region, shown as i5 or i7, that varies in sequence between different samples of a multiplexed sample. Other samples in the run can be prepared with primers that include their respective indexes. Accordingly, certain sequence reads may be obtained with universal primers while other sequence reads are obtained with primers or a mix of primers that are specific to indexes of one or more samples in a multiplexed reaction.

In an embodiment, the adapter sequence may include CS1 Common sequence 1 (CS1) (5′-ACACTGACGACATGGTTCTACA-3′) (SEQ ID NO: 21) or common sequence 2 (CS2) (5′-TACGGTAGCAGAGACTTGGTCT-3′) (SEQ ID NO: 22), which are universal primer sequences for Illumina MiSeq amplicon tagging and indexing. It should be understood that the generated sequencing library may include double-stranded fragments with disclosed adapter sequences on one strand and complementary sequences on an opposing strand.

In an embodiment, unique molecular identifiers (UMIs) may be incorporated into oligonucleotides as provided herein, e.g., via ligation. UMIs are short sequences used to uniquely tag each molecule in a sample library to provide error correction and reduce sequencing bias.

FIG. 14 is a schematic diagram of a sequencing device 500 that may be used in conjunction with the disclosed embodiments for acquiring sequencing data of identification sequences and/or index sequences as generally discussed herein. The sequence device 500 may be implemented according to any sequencing technique, such as those incorporating sequencing-by-synthesis methods described in U.S. Patent Publication Nos. 2007/0166705; 2006/0188901; 2006/0240439; 2006/0281109; 2005/0100900; U.S. Pat. No. 7,057,026; WO 05/065814; WO 06/064199; WO 07/010,251, the disclosures of which are incorporated herein by reference in their entireties. Alternatively, sequencing by ligation techniques may be used in the sequencing device 500. Such techniques use DNA ligase to incorporate oligonucleotides and identify the incorporation of such oligonucleotides and are described in U.S. Pat. Nos. 6,969,488; 6,172,218; and 6,306,597; the disclosures of which are incorporated herein by reference in their entireties. Some embodiments can utilize nanopore sequencing, whereby target nucleic acid strands, or nucleotides exonucleolytically removed from target nucleic acids, pass through a nanopore. As the target nucleic acids or nucleotides pass through the nanopore, each type of base can be identified by measuring fluctuations in the electrical conductance of the pore (U.S. Pat. No. 7,001,792; Soni & Meller, Clin. Chem. 53, 1996-2001 (2007); Healy, Nanomed. 2, 459-481 (2007); and Cockroft, et al. J. Am. Chem. Soc. 130, 818-820 (2008), the disclosures of which are incorporated herein by reference in their entireties). Yet other embodiments include detection of a proton released upon incorporation of a nucleotide into an extension product. For example, sequencing based on detection of released protons can use an electrical detector and associated techniques that are commercially available from Ion Torrent (Guilford, CT, a Life Technologies subsidiary) or sequencing methods and systems described in US 2009/0026082 A1; US 2009/0127589 A1; US 2010/0137143 A1; or US 2010/0282617 A1, each of which is incorporated herein by reference in its entirety. Particular embodiments can utilize methods involving the real-time monitoring of DNA polymerase activity. Nucleotide incorporations can be detected through fluorescence resonance energy transfer (FRET) interactions between a fluorophore-bearing polymerase and γ-phosphate-labeled nucleotides, or with zeromode waveguides as described, for example, in Levene et al. Science 299, 682-686 (2003); Lundquist et al. Opt. Lett. 33, 1026-1028 (2008); Korlach et al. Proc. Natl. Acad. Sci. USA 105, 1176-1181 (2008), the disclosures of which are incorporated herein by reference in their entireties. Other suitable alternative techniques include, for example, fluorescent in situ sequencing (FISSEQ), and Massively Parallel Signature Sequencing (MPSS). In particular embodiments, the sequencing device 500 may be a HiSeq, MiSeq, or HiScanSQ from Illumina (La Jolla, CA). In other embodiment, the sequencing device 500 may be configured to operate using a CMOS sensor with nanowells fabricated over photodiodes such that DNA deposition is aligned one-to-one with each photodiode.

The sequencing device 500 may be “one-channel” a detection device, in which only two of four nucleotides are labeled and detectable for any given image. For example, thymine may have a permanent fluorescent label, while adenine uses the same fluorescent label in a detachable form. Guanine may be permanently dark, and cytosine may be initially dark but capable of having a label added during the cycle. Accordingly, each cycle may involve an initial image and a second image in which dye is cleaved from any adenines and added to any cytosines such that only thymine and adenine are detectable in the initial image but only thymine and cytosine are detectable in the second image. Any base that is dark through both images in guanine and any base that is detectable through both images is thymine. A base that is detectable in the first image but not the second is adenine, and a base that is not detectable in the first image but detectable in the second image is cytosine. By combining the information from the initial image and the second image, all four bases are able to be discriminated using one channel.

In the depicted embodiment, the sequencing device 500 includes a separate sample processing device 502 and an associated computer 504. However, as noted, these may be implemented as a single device. Further, the associated computer 504 may be local to or networked or otherwise in communication with the sample processing device 502. In the depicted embodiment, the biological sample may be loaded into the sample processing device 502 on a sample substrate 510, e.g., a flow cell or slide, that is imaged to generate sequence data. For example, reagents that interact with the biological sample fluoresce at particular wavelengths in response to an excitation beam generated by an imager 512 and thereby return radiation for imaging. For instance, the fluorescent components may be generated by fluorescently tagged nucleic acids that hybridize to complementary molecules of the components or to fluorescently tagged nucleotides that are incorporated into an oligonucleotide using a polymerase. As will be appreciated by those skilled in the art, the wavelength at which the dyes of the sample are excited and the wavelength at which they fluoresce will depend upon the absorption and emission spectra of the specific dyes. Such returned radiation may propagate back through the directing optics. This retrobeam may generally be directed toward detection optics of the imager 512.

The imager detection optics may be based upon any suitable technology, and may be, for example, a charged coupled device (CCD) sensor that generates pixilated image data based upon photons impacting locations in the device. However, it will be understood that any of a variety of other detectors may also be used including, but not limited to, a detector array configured for time delay integration (TDI) operation, a complementary metal oxide semiconductor (CMOS) detector, an avalanche photodiode (APD) detector, a Geiger-mode photon counter, or any other suitable detector. TDI mode detection can be coupled with line scanning as described in U.S. Pat. No. 7,329,860, which is incorporated herein by reference. Other useful detectors are described, for example, in the references provided previously herein in the context of various nucleic acid sequencing methodologies.

The imager 512 may be under processor control, e.g., via a processor 514, and the sample receiving device 502 may also include I/O controls 516, an internal bus 518, non-volatile memory 520, RAM 522 and any other memory structure such that the memory is capable of storing executable instructions, and other suitable hardware components that may be similar to those described with regard to FIG. 31. Further, the associated computer 504 may also include a processor 524, I/O controls 526, communications circuitry 527, and a memory architecture including RAM 528 and non-volatile memory 530, such that the memory architecture is capable of storing executable instructions 532. The hardware components may be linked by an internal bus, which may also link to the display 534. In embodiments in which the sequencing device 500 is implemented as an all-in-one device, certain redundant hardware elements may be eliminated.

The processor 514, 524 may be programmed to assign individual sequencing reads to a sample based on the associated index sequence or sequences according to the techniques provided herein. In particular embodiments, based on the image data acquired by the imager 512, the sequencing device 500 may be configured to generate sequencing data that includes base calls for each base of a sequencing read. Further, based on the image data, even for sequencing reads that are performed in series, the individual reads may be linked to the same location via the image data and, therefore, to the same template strand. In this manner, index sequencing reads may be associated with a sequencing read of an insert sequence before being assigned to a sample of origin. The processor 514, 524 may also be programmed to perform downstream analysis on the sequences corresponding to the inserts for a particular sample subsequent to assignment of sequencing reads to the sample.

In certain embodiments, the I/O controls 516, 526 may be configured to receive user inputs that automatically select sequencing parameters based on the associated sequence library preparation techniques. In an embodiment, the user input may be a selection of a sequence library preparation kit or reading a barcode or identifier of a sequence library preparation kit.

In embodiments of the disclosed techniques, aptamer detection may be based on a presence of the uniquely identifying identification sequence 64 for an individual aptamer 20 in sequencing data generated by the sequencing device 500. Accordingly, in an embodiment, the sequencing device 500 may perform analysis of sequence reads to identify one or more identification sequences 64 for a panel of aptamers 20. In an embodiment, all or part of the aptamer 20 may be directly sequenced to identify the aptamer 20. Based on the identified aptamers 20, a notification or report of positive aptamer identification may be generated. In an embodiment, the notification is provided on the display 534 or communicated via the communications circuitry 527 to a remote device or a cloud server.

This written description uses examples to enable any person skilled in the art to practice the disclosed embodiments, including making and using any devices or systems and performing any incorporated methods. The patentable scope is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A method of aptamer detection, comprising:
contacting analytes of a sample with a plurality of aptamers under conditions that permit analyte-aptamer complexes to form, wherein different aptamers of the plurality of aptamers have specific affinity for respective different analytes of the analytes; and
detecting the analytes by detecting aptamers of the analyte-aptamer complexes, wherein detecting an individual aptamer of the plurality of aptamers comprises:
contacting the individual aptamer with an exonuclease to deprotect a 3′ end of the individual aptamer to generate a deprotected 3′ end of the individual aptamer;
modifying the deprotected 3′ end of the individual aptamer to generate a modified 3′end of the individual aptamer;
capturing the individual aptamer using the modified 3′ end; and
detecting the captured individual aptamer.

2. The method of claim 1, wherein modifying the deprotected 3′ end comprises:
hybridizing a first portion of a first oligonucleotide to a region of the individual aptamer comprising the deprotected 3′ end; and
ligating a second oligonucleotide to the deprotected 3′ end, wherein the second oligonucleotide hybridizes to a second portion of the first oligonucleotide, and wherein the second oligonucleotide comprises an affinity tag.

3. The method of claim 2, wherein the individual aptamer is captured via an affinity tag capture molecule.

4. The method of claim 3, wherein the affinity tag is biotin and the affinity tag capture molecule is avidin or streptavidin.

5. The method of claim 3, wherein the affinity tag capture molecule is coupled to a bead or substrate.

6. The method of claim 2, wherein the second oligonucleotide comprises an adapter sequence.

7. The method of claim 1, wherein detecting the captured individual aptamer comprises sequencing the captured individual aptamer.

8. The method of claim 1, wherein detecting the captured individual aptamer comprises generating a sequencing library comprising one or more amplification products of the captured individual aptamer.

9. The method of claim 1, further comprising washing unbound first oligonucleotides from the captured individual aptamer.

10. The method of claim 1, wherein modifying the deprotected 3′ end of the individual aptamer to generate a modified 3′end of the individual aptamer comprises incorporating an oligo-linked nucleotide (oNTP) onto the deprotected 3′ end of the individual aptamer.

11. The method of claim 1, wherein modifying the deprotected 3′ end of the individual aptamer to generate a modified 3′end of the individual aptamer comprises incorporating a biotin-tagged nucleotide onto the deprotected 3′ end of the individual aptamer.

12. The method of claim 11, further comprising hybridizing a reporter probe to the individual aptamer, wherein the reporter probe comprises an identification sequence flanked by conserved primer sequences.

13. The method of claim 12, wherein detecting the individual aptamer comprises amplifying the identification sequence using the conserved primer sequences or sequences complementary to the conserved primer sequences.

14. A method of aptamer detection, comprising:
contacting analytes of a sample with a plurality of aptamers under conditions that permit analyte-aptamer complexes to form, wherein different aptamers of the plurality of aptamers have specific affinity for respective different analytes of the analytes; and
detecting the analytes by detecting aptamers of the analyte-aptamer complexes, wherein detecting an individual aptamer of the plurality of aptamers comprises:
modifying a 3′ end of the individual aptamer to generate a modified 3′end of the individual aptamer;
hybridizing an oligonucleotide to the individual aptamer, wherein the oligonucleotide comprises a nonhybridizing 5′ region;
extending an oligonucleotide 3′ end to generate an extended strand; and
using the extended strand to generate a fragment of a sequencing library.

15.-23. (canceled)

24. An aptamer detection probe set, comprising:
a plurality of different probe mixtures complementary to respective different aptamers of an aptamer panel, wherein an individual probe mixture of the plurality of different probe mixtures comprises:
a binding subset of probes coupled to an affinity tag; and
a dummy subset of probes not coupled to the affinity tag, wherein each probe in the binding subset and the dummy subset of the individual probe mixture comprises a same binding region that is complementary to at least a portion of an individual aptamer of the aptamer panel and wherein each probe in the binding subset and the dummy subset of the individual probe mixture comprises a nonhybridizing region at a 3′ end.

25.-27. (canceled)

28. An aptamer detection probe set, comprising:
a plurality of different first probe mixtures complementary to respective different aptamers of an aptamer panel, wherein an individual probe mixture of the plurality of different first probe mixtures comprises:
a binding subset of probes; and
a dummy subset of probes comprising a modified 5′ end that cannot be ligated, wherein each probe in the binding subset and the dummy subset of the individual probe mixture comprises a same binding region that is complementary to at least a portion of an individual aptamer of the aptamer panel and wherein the binding subset of probes have an unmodified 5′ end that is capable of being ligated.

29. A method of aptamer detection, comprising:
contacting analytes of a sample with a plurality of aptamers under conditions that permit analyte-aptamer complexes to form, wherein different aptamers of the plurality of aptamers have specific affinity for respective different analytes of the analytes; and
detecting the analytes by detecting aptamers of the analyte-aptamer complexes, wherein detecting an individual aptamer of the plurality of aptamers comprises:
contacting the individual aptamer with a single-stranded nucleic acid reporter probe to form an aptamer-reporter probe complex, the reporter probe comprising:
an aptamer binding region that binds to the individual aptamer to form a first double-stranded region; and
a single-stranded region comprising a cleavage region and an identification sequence uniquely identifying for the individual aptamer or an associated analyte;
extending the individual aptamer from a 3′ end to form a second double-stranded region comprising the identification sequence and using the single-stranded region as a template;
separating the first double-stranded region from the second double-stranded region at the cleavage region; and
sequencing the second double-stranded region to detect the identification sequence.

30.-45. (canceled)

46. An aptamer detection reporter probe set, comprising:
a plurality of different single-stranded nucleic acid reporter probes complementary to respective different aptamers of an aptamer panel,
wherein a first single-stranded nucleic acid reporter probe of the plurality of different single-stranded nucleic acid reporter probes comprises:
a first aptamer binding region that binds to a first individual aptamer to form a first double-stranded region;
a first cleavage region; and
a first identification sequence uniquely identifying for the first individual aptamer; and
wherein a second single-stranded nucleic acid reporter probe of the plurality of different single-stranded nucleic acid reporter probes comprises:
a second aptamer binding region that binds to a second individual aptamer to form a second double-stranded region;
a second cleavage region; and
a second identification sequence uniquely identifying for the second individual aptamer; wherein the first aptamer binding region and the second aptamer binding region have different nucleotide sequences relative to one another.

47.-54. (canceled)

55. A method of aptamer detection, comprising:
contacting an individual aptamer with a reporter probe that hybridizes to the individual aptamer at an aptamer binding region to form a first double-stranded region of the reporter probe and wherein the reporter probe comprises a nonhybridizing region comprising a cleavage region and an identification sequence uniquely identifying for the individual aptamer or an associated analyte;
extending the individual aptamer from a 3′ end to form a second double-stranded region using the non-hybridizing region as a template;
separating the first double-stranded region from the second double-stranded region at the cleavage region; and
detecting the identification sequence.

59. A method of aptamer detection, comprising:
contacting analytes of a sample with a plurality of aptamers under conditions that permit analyte-aptamer complexes to form, wherein different aptamers of the plurality of aptamers have specific affinity for respective different analytes of the analytes; and
detecting the analytes by detecting aptamers of the analyte-aptamer complexes, wherein detecting an individual aptamer of the plurality of aptamers comprises:
contacting the individual aptamer with an exonuclease to deprotect a 3′ end of the individual aptamer to generate a deprotected 3′ end of the individual aptamer;
extending the deprotected 3′ end of the individual aptamer using a polymerase to generate an extended 3′end of the individual aptamer;
hybridizing the individual aptamer to a reporter probe, wherein the reporter probe has a protected 3′ end that is not deprotected by the exonuclease or extended by the polymerase;
capturing the individual aptamer using the modified 3′ end; and
detecting the captured individual aptamer using the reporter probe.

60.-61. (canceled)


SEQUENCE LISTING (22 sequences)

SEQ ID NO: 1 - DNA, 33 bp, synthetic construct
  misc_feature 1..33: note = Synthetic: A14-ME
  source 1..33: mol_type = other DNA
tcgtcggcagcgtcagatgtgtataagagacag

SEQ ID NO: 2 - DNA, 34 bp, synthetic construct
  misc_feature 1..34: note = Synthetic: B1ME
  source 1..34: mol_type = other DNA
gtctcgtgggctcggagatgtgtataagagacag

SEQ ID NO: 3 - DNA, 19 bp, synthetic construct
  misc_feature 1..19: note = Synthetic: ME'
  modified_base 1: mod_base = OTHER; note = Phosphate
  source 1..19: mol_type = other DNA
ctgtctcttatacacatct

SEQ ID NO: 4 - DNA, 14 bp, synthetic construct
  misc_feature 1..14: note = Synthetic: A14
  source 1..14: mol_type = other DNA
tcgtcggcagcgtc

SEQ ID NO: 5 - DNA, 15 bp, synthetic construct
  misc_feature 1..15: note = Synthetic: B15
  source 1..15: mol_type = other DNA
gtctcgtgggctcgg

SEQ ID NO: 6 - DNA, 19 bp, synthetic construct
  misc_feature 1..19: note = Synthetic: ME
  source 1..19: mol_type = other DNA
agatgtgtataagagacag

SEQ ID NO: 7 - DNA, 20 bp, synthetic construct
  misc_feature 1..20: note = Synthetic: P5
  source 1..20: mol_type = other DNA
aatgatacggcgaccaccga

SEQ ID NO: 8 - DNA, 21 bp, synthetic construct
  misc_feature 1..21: note = Synthetic: P7
  source 1..21: mol_type = other DNA
caagcagaagacggcatacga

SEQ ID NO: 9 - DNA, 20 bp, synthetic construct
  misc_feature 1..20: note = Synthetic: anti-P5
  source 1..20: mol_type = other DNA
tcggtggtcgccgtatcatt

SEQ ID NO: 10 - DNA, 21 bp, synthetic construct
  misc_feature 1..21: note = Synthetic: anti-P7
  source 1..21: mol_type = other DNA
tcgtatgccgtcttctgcttg

SEQ ID NO: 11 - DNA, 33 bp, synthetic construct
  misc_feature 1..33: note = Synthetic: SBS3
  source 1..33: mol_type = other DNA
acactctttccctacacgacgctcttccgatct

SEQ ID NO: 12 - DNA, 37 bp, synthetic construct
  misc_feature 1..37: note = Synthetic: SBS8
  source 1..37: mol_type = other DNA
cggtctcggcattcctgctgaaccgctcttccgatct

SEQ ID NO: 13 - DNA, 33 bp, synthetic construct
  misc_feature 1..33: note = Synthetic: anti-SBS3
  source 1..33: mol_type = other DNA
agatcggaagagcgtcgtgtagggaaagagtgt

SEQ ID NO: 14 - DNA, 37 bp, synthetic construct
  misc_feature 1..37: note = Synthetic: anti-SBS8
  source 1..37: mol_type = other DNA
agatcggaagagcggttcagcaggaatgccgagaccg

SEQ ID NO: 15 - DNA, 33 bp, synthetic construct; identical to SEQ ID NO: 1
  misc_feature 1..33: note = Synthetic: Read 1
  source 1..33: mol_type = other DNA

SEQ ID NO: 16 - DNA, 34 bp, synthetic construct; identical to SEQ ID NO: 2
  misc_feature 1..34: note = Synthetic: Read 2
  source 1..34: mol_type = other DNA

SEQ ID NO: 17 - DNA, 33 bp, synthetic construct; identical to SEQ ID NO: 11
  misc_feature 1..33: note = Synthetic: Paired End Read 1
  source 1..33: mol_type = other DNA

SEQ ID NO: 18 - DNA, 37 bp, synthetic construct; identical to SEQ ID NO: 12
  misc_feature 1..37: note = Synthetic: Paired End Read 2
  source 1..37: mol_type = other DNA

SEQ ID NO: 19 - DNA, 39 bp, synthetic construct
  misc_feature 1..39: note = Synthetic: Index 1 Read
  misc_feature 24..25: note = [i7] site
  source 1..39: mol_type = other DNA
caagcagaagacggcatacgagatgtctcgtgggctcgg

SEQ ID NO: 20 - DNA, 43 bp, synthetic construct
  misc_feature 1..43: note = Synthetic: Index 2 Read
  misc_feature 29..30: note = [i5] site
  source 1..43: mol_type = other DNA
aatgatacggcgaccaccgagatctacactcgtcggcagcgtc

SEQ ID NO: 21 - DNA, 22 bp, synthetic construct
  misc_feature 1..22: note = Synthetic: CS1
  source 1..22: mol_type = other DNA
acactgacgacatggttctaca

SEQ ID NO: 22 - DNA, 22 bp, synthetic construct
  misc_feature 1..22: note = Synthetic: CS2
  source 1..22: mol_type = other DNA
tacggtagcagagacttggtct